(19) United States
(12) Patent Application Publication
(10) Pub. No.: US 2019/0101525 A1
(43) Pub. Date: Apr. 4, 2019

(54) METHOD FOR DETERMINING THE EXTENT OF AGING OF A SUBJECT AND TREATING THE SUBJECT TO SUPPRESS THE EXTENT OF AGING

(71) Applicant: OKINAWA INSTITUTE OF SCIENCE AND TECHNOLOGY SCHOOL CORPORATION, Okinawa (JP)

(72) Inventors: Mitsuhiro Yanagida, Okinawa (JP); Hiroshi Kondoh, Kyoto (JP); Takayuki Teruya, Okinawa (JP)

(73) Assignee: OKINAWA INSTITUTE OF SCIENCE AND TECHNOLOGY SCHOOL CORPORATION, Okinawa (JP)

(21) Appl. No.: 16/127,801

(22) Filed: Sep. 11, 2018

Related U.S. Application Data
(63) Continuation-in-part of application No. PCT/JP2017/009758, filed on Mar. 10, 2017.
(60) Provisional application No. 62/366,803, filed on Jul. 26, 2016.
(60) Provisional application No. 62/307,092, filed on Mar. 11, 2016.

(51) Int. Cl. G01N 33/49; G01N 33/68; G01N 33/92

(52) U.S. Cl. CPC G01N 33/492; G01N 33/6848; G01N 2800/60; G01N 2800/7042; G01N 33/92

(57) ABSTRACT

A method for suppressing aging includes obtaining a blood sample from a subject, detecting one or more blood metabolites in the blood sample, identifying the subject as having a biological age greater than a chronological age of the subject by determining metabolite levels of the one or more blood metabolites of the subject, and subjecting the identified subject to fasting more than 10 hours.

### TECHNICAL FIELD

One or more embodiments of the present invention relate to a method for determining the extent of aging of a subject and treating the subject to suppress the extent of aging, a system for determining the extent of aging, a kit for determining the extent of aging and a method of evaluating substances which affect the extent of aging.

### BACKGROUND

Human blood metabolites have been well investigated to determine their abundance and biological significance, and for their potential use as diagnostic markers. For medical diagnosis, non-cellular metabolites from plasma or serum, are mostly commonly employed due to the simplicity in collecting and examining them. While mature human red blood cells (RBCs) lack nuclei and cellular organelles (NPL 1), RBCs utilize glycolysis for ATP production, maintain redox homeostasis, and osmoregulate (NPL 2). Their active metabolism supports cellular homeostasis and ensures lifespans of ˜4 months (NPL 3). Their metabolites may reflect health status or environmental stresses differently than do metabolites of plasma. As RBCs occupy about half the total blood volume (ca. 5 L), their metabolite profiles, which have scarcely been investigated, seemed worthy of investigation.

Metabolomics is a branch of chemical biology that profiles metabolites in cells and organisms, using techniques such as liquid chromatography (LC)-mass spectrometry (MS). It usually deals with molecules <1.5 kDa, and is an important tool for studying metabolic regulation in combination with other comprehensive analyses, such as proteomics and transcriptomics. Recently we reported that among 133 compounds identified in human blood, 101 are also found in the fission yeast, Schizosaccharomyces pombe (NPL 4), implying that many metabolites might be evolutionarily conserved. Quantitative measurements of an array of compounds among individuals, offer profound insights into health or disease conditions and effects of nutrition, drugs, and stress. Moreover, comprehensive information about individual variation in metabolites could impact the future of medical science (NPL 5-11).

While blood consists of noncellular (plasma or serum) and cellular components, most human blood metabolomics studies have focused on plasma or serum, for which large biobanks (curated collections of samples of plasma, urine, etc.) are now available (12-16). These studies are useful to understand disease mechanisms and to identify diagnostic markers for diseases, such as diabetes (NPL 17). Some genome-wide studies have also employed metabolomics (reviewed in Kastenmueller et al 2015 (NPL 18)). In contrast, few comprehensive metabolomics reports exist regarding red blood cells (RBCs) [e.g. Nishino et al 2009 (NPL 19)], although RBCs comprise nearly half the blood volume. This is partly due to technical difficulties in stabilizing labile cellular metabolites (NPL 20).

### CITATION LIST

### Non Patent Literature

[NPL 1]
Rapoport S M, Schewe T, & Thiele B-J (1990) Maturational breakdown of mitochondria and other organelles in reticulocytes. in Erythroid Cells, ed Harris J R (Springer US), pp 151-194.
[NPL 2]
van Wijk R & van Solinge W W (2005) The energy-less red blood cell is lost: erythrocyte enzyme abnormalities of glycolysis. Blood 106(13):4034-4042.
[NPL 3]
Bax B E, Bain M D, Talbot P J, Parker-Williams E J, & Chalmers R A (1999) Survival of human carrier erythrocytes in vivo. Clin Sci (Lond) 96(2):171-178.
[NPL 4]
Chaleckis R, et al. (2014) Unexpected similarities between the Schizosaccharomyces and human blood metabolomes, and novel human metabolites. Molecular BioSystems 10(10):2538.
[NPL 5]
Fernie A R, Trethewey R N, Krotzky A J, & Willmitzer L (2004) Metabolite profiling: from diagnostics to systems biology. Nat Rev Mol Cell Biol 5(9):763-769.
[NPL 6]
Goodacre R, Vaidyanathan S, Dunn W B, Harrigan G G, & Kell D B (2004) Metabolomics by numbers: acquiring and understanding global metabolite data. Trends Biotechnol 22(5):245-252.
[NPL 7]
Hirai M Y, et al. (2004) Integration of transcriptomics and metabolomics for understanding of global responses to nutritional stresses in Arabidopsis thaliana. Proc Natl Acad Sci USA 101(27): 10205-10210.
[NPL 8]
Kell D B (2004) Metabolomics and systems biology: making sense of the soup. Curr Opin Microbiol 7(3):296-307.
[NPL 9]
Nicholson J K & Lindon J C (2008) Systems biology: Metabonomics. Nature 455(7216): 1054-1056.
[NPL 10]
Patti G J, Yanes O, & Siuzdak G (2012) Innovation: Metabolomics: the apogee of the omics trilogy. Nat Rev Mol Cell Biol 13(4):263-269.
[NPL 11]
Ramautar R, Berger R, van der Greef J, & Hankemeier T (2013) Human metabolomics: strategies to understand biology. Curr Opin Chem Biol 17(5):841-846.
[NPL 12]
Dunn W B, et al. (2015) Molecular phenotyping of a UK population: defining the human serum metabolome. Metabolomics 11:9-26.
[NPL 13]
Guertin K A, et al. (2014) Metabolomics in nutritional epidemiology: identifying metabolites associated with diet and quantifying their potential to uncover diet-disease relations in populations. Am J Clin Nutr 100(1):208-217.
[NPL 14]
Lawton K A, et al. (2008) Analysis of the adult human plasma metabolome. Pharmacogenomics 9(4):383-397.
[NPL 15]
Psychogios N, et al. (2011) The human serum metabolome. PLoS One 6(2):e16957.
[NPL 16]
Yu Z, et al. (2012) Human serum metabolic profiles are age dependent. Aging Cell 11(6):960-967.
[NPL 17]
Suhre K (2014) Metabolic profiling in diabetes. J Endocrinol 221(3):R75-85.
[NPL 18]
Kastenmuller G, Raffler J, Gieger C, & Suhre K (2015) Genetics of human metabolism: an update. Hum Mol Genet 24(R1):R93-R101.
[NPL 19]
Nishino T, et al. (2009) In silico modeling and metabolome analysis of long-stored erythrocytes to improve blood storage methods. J Biotechnol 144(3):212-223.
[NPL 20]
Gil A, et al. (2015) Stability of energy metabolites—An often overlooked issue in metabolomics studies: A review. Electrophoresis 36(18):2156-2169.
[NPL 21]
Pluskal T, Nakamura T, Villar-Briones A, & Yanagida M (2010) Metabolic profiling of the fission yeast S. pombe: quantification of compounds under different temperatures and genetic perturbation. Mol Biosyst 6(1): 182-198.

One or more embodiments of the present invention provide a novel method capable of simply and accurately determining the extent of aging. One or more embodiments of the present invention also provide a method for treating the subject to suppress the extent of aging.

### SUMMARY

Metabolites present in human blood document individual physiological states influenced by genetic, epigenetic, and life-style factors. Using high-resolution liquid chromatography-mass spectrometry (LC-MS), we performed non-targeted, quantitative metabolomics analysis in blood of 15 young (29±4 yr) and 15 elderly (81±7 yr) individuals. Coefficients of variation (CV=standard deviation/mean) were obtained for 126 blood metabolites of all 30 donors. Fifty-five RBC-enriched metabolites, for which metabolomics studies have been scarce, are highlighted here. We found forty three blood compounds that show remarkable age-related increases or decreases. Eighteen of them are RBC-enriched, suggesting that RBC metabolomics is highly valuable for human aging research. Age differences are partly explained by a decrease in anti-oxidant production or increasing inefficiency of urea metabolism among the elderly. Pearson's coefficients demonstrated that some age-related compounds are correlated, suggesting that aging affects them concomitantly. While our CV values are mostly consistent with those previously published, we here report novel CVs of 51 blood compounds. Compounds having moderate to high CV values (0.4-2.5) are often modified. Compounds having low CV values such as ATP and glutathione may be related to various diseases as their concentrations are strictly controlled, and changes in them would compromise health. Thus human blood is a rich source of information about individual metabolic differences.

Fasting is one of the most significant physiological stimuli to the human body, as nutrient limitation greatly affects energy production, triggering a wide range of catabolic reactions. The body's glycogen storage capacity is limited and rapidly exhausted, and nutrients such as lipids are consumed as energy substitutes for glucose, which under non fasting conditions, is employed as the major fuel source. After glycogen stores are depleted, gluconeogenesis is employed to maintain blood sugar levels. Radioisotope experiments have shown that constitutively activated gluconeogenesis accounts for the majority of glucose production in human body after prolonged fasting (Rothman, D. L., Magnusson, I., Katz, L. D., Shulman, R. G. & Shulman, G. I. Quantitation of Hepatic Glycogenolysis and Gluconeogenesis in Fasting Humans with C-13 Nmr. Science 254, 573-576 (1991), Landau, B. R. et al. Contributions of gluconeogenesis to glucose production in the fasted state. J. Clin. Invest. 98, 378-385 (1996)). In addition to gluconeogenesis, evidence from serum or plasma suggests that fasting stress forces the human body to utilize various non-glucose metabolites, such as conversion of 3-hydroxybutyrate (3-HB) into acetyl-CoA, as energy sources (Owen, O. E., Felig, P., Morgan, A. P., Wahren, J. & Cahill, G. F. Liver and Kidney Metabolism during Prolonged Starvation. J. Clin. Invest. 48, 574-& (1969), Cahill, G. F., Jr. Fuel metabolism in starvation. Annu. Rev. Nutr. 26, 1-22 (2006)).

We analyzed metabolites during fasting, to monitor their changes. As most metabolic studies of fasting have tracked specific plasma or serum metabolites, such as branched-chain amino acids (BCAAs), our exhaustive, non-targeted analysis was intended to identify new fasting marker metabolites. Interestingly, we found that some blood metabolites which are lower in elder than in young are upregulated by fasting.

In this work, we present a novel method for determining the extent of aging in which a blood metabolite is used as an indicator. The method according to one or more embodiments of the present invention is easy and accurate. We also present a novel method for treating the subject to suppress the extent of aging.

One or more embodiments of the present inventions include the following:

[1] A method for determining the extent of aging of a subject and treating the subject to suppress the extent of aging, said method comprising the steps of;
(a) detecting the extent of aging of a subject by using a blood metabolite as an indicator; and
(b) having the subject fast more than 10 hours.
[2] The method according to [1], wherein at least one selected from the group consisting of whole blood, Red blood cells and plasma from a subject is used as a sample, and a blood metabolite in the sample is used as an indicator.
[3] The method according to [2], wherein the sample is treated with cold organic solvent immediately after bleeding.
[4] The method according to [3], wherein the blood metabolite comprises at least one metabolite selected from the group consisting of Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, Leucine, N2-Acetyl-lysine, Ophthalmic acid, Pantothenate, N6-Acetyl-lysine, NAD+, CDP-choline, Glycerophosphocholine, Histidine, Phenylalanine, Phosphocreatine, Tyrosine, Isoleucine, NADP+, Pentose-phosphate, S-Adenosyl-homocysteine, CDP-ethanolamine, Creatine, CTP, Fructose-6-phosphate, Glycerol-phosphate, Serine, Tryptophan, UDP-glucose, Adenosine, Aspartate, Dimethyl-arginine, Diphospho-glycerate, Glucose-6-phosphate, Glutamate, Glutarate, N-Acetyl-(iso)leucine, and Ketovaline.
[5] The method according to [3], wherein the blood metabolite comprises at least one metabolite selected from the group consisting of Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, Leucine, N2-Acetyl-lysine, Ophthalmic acid, Pantothenate, N6-Acetyl-lysine, NAD+, CDP-choline, Glycerophosphocholine, Histidine, Phenylalanine, Phosphocreatine, Tyrosine, Isoleucine, NADP+, Pentose-phosphate, Ketovaline and S-Adenosyl-homocysteine.
[6] The method according to [3], wherein the blood metabolite comprises at least one metabolite selected from the group consisting of Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, Leucine, Ophthalmic acid, Isoleucine, and Ketovaline.
[7] The method according to [3], wherein the blood metabolite comprises at least one metabolite selected from the group consisting of Keto(iso)leucine, Leucine, Ophthalmic acid, Isoleucine, and Ketovaline.
[8] The method according to [4], wherein the subject fasts more than 20 hours in step (b).
[9] An apparatus for determining the extent of aging, wherein the extent of aging is determined by the method according to [1].
[10] An apparatus for determining the extent of aging which comprises means for input and means for determining, wherein data of blood metabolites of the subject are input to the means for input, and the extent of aging is determined by comparing the data of the subject and the data of the population.
[11] A system for determining the extent of aging in which the extent of aging is determined by the method according to any one of claims claim 1.
[12] A method of evaluating substances which affect the extent of aging comprising the step of measuring a blood metabolite, wherein the blood metabolite comprises at least one metabolite selected from the group consisting of Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, Leucine, N2-Acetyl-lysine, Ophthalmic acid, Pantothenate, N6-Acetyl-lysine, NAD+, CDP-choline, Glycerophosphocholine, Histidine, Phenylalanine, Phosphocreatine, Tyrosine, Isoleucine, NADP+, Pentose-phosphate, S-Adenosyl-homocysteine, CDP-ethanolamine, Creatine, CTP, Fructose-6-phosphate, Glycerol-phosphate, Serine, Tryptophan, UDP-glucose, Adenosine, Aspartate, Dimethyl-arginine, Diphospho-glycerate, Glucose-6-phosphate, Glutamate, Glutarate, N-Acetyl-(iso)leucine, and Ketovaline.
[13] A kit for determining the extent of aging by using the methods according to any one of claim 1, comprising blood collection tubes and blood metabolite compounds as detection standard.

Human blood provides a rich source of information about metabolite that reflects individual differences in health, disease, diet and life-style. The coefficient of variation for human blood metabolites enriched in red blood cells or plasma were quantified after careful preparation. We were able to identify 43 age-related metabolites. Metabolites that decline strikingly in elderly include anti-oxidants and those involved in high physical activity. Metabolites that increase significantly in elderly include those related to declining renal and liver function. Statistical analysis suggests that certain age-related compounds either increased or decreased in the elderly are correlated. Individual variability in blood metabolites may lead to identify candidates for markers of human aging or relevant diseases. One or more embodiments of the present invention provide a novel method capable of simply and accurately determining the extent of aging. We also found that some blood metabolites which are lower in elder than in young are upregulated by fasting. Thus one or more embodiments of the present invention provide a novel method for suppressing the extent of aging of the subject in need.

### BRIEF DESCRIPTION OF DRAWINGS

FIGS. 1A and 1B show CV profiles for 126 human blood metabolites. FIG. 1A shows a summary of 126 blood compounds with coefficients of variation (CV₃₀) in six different ranges. Upper panel, <0.3, 0.3˜0.4; Lower panel, 0.4˜0.5, 0.5˜0.7, 0.7˜1.0, 1.0˜2.5). The lowest CV₃₀ (<0.3) group contains 28 compounds. RBC-enriched compounds are highlighted in gray. Abundance of compounds indicated by their peak areas: red, compounds with high peak areas (>108 AU); green, medium peak areas (108˜107 AU); blue, with low peak areas (<107 AU). Compounds for which CVs have not previously been reported in the literature are underlined. The number in the blue box represents all compounds listed in one CV range, while the number in the red box represents compounds for which CVs reported here are new. FIG. 1B shows an overview of compound numbers in low and high variability groups.

FIGS. 2A to 2C show clusters of human blood metabolites, defined by structure or function, showing similar CVs. Blood data from all 30 volunteers revealed several groups of compounds with Pearson correlation coefficients >0.7. Among these clusters were compounds related to ergothioneine (FIG. 2A), glycolytic pathway metabolites (FIG. 2B), and methylated compounds (FIG. 2C). Pearson correlation coefficients between pairs of compounds are shown in the upper right corners of the panels. In the lower left corners, actual compound levels are plotted for each pair.

FIGS. 3A to 3H are graphs showing that essential metabolites are almost invariant, while modified metabolites (e.g., methylated amino acids) vary widely. Distributions of ATP (FIG. 3A), glutathione disulfide (GSSG) (FIG. 3B), diphosphoglycerate (FIG. 3C), glucose-6-phosphate (FIG. 3D), trimethyl-histidine (FIG. 3E), UDP-acetyl-glucosamine (FIG. 3F), 4-guanidinobutanoate (FIG. 3G), trimethyl-tryptophan (FIG. 3H) in blood of 30 individuals. Black, orange, and azure dots represent all, elderly, and young subjects, respectively. Peak areas of metabolites were divided into 10 bins in each group. Error bars represent means±SD.

FIGS. 4A to 4H are graphs showing identification of some blood metabolites that differ in abundance between young (29±4 years) and elderly (81±7) persons. 1,5-Anhydroglucitol (FIG. 4A), ophthalmic acid (FIG. 4B), acetyl-carnosine (FIG. 4C), and carnosine (FIG. 4D) are higher in young subjects, while citrulline (FIG. 4E), pantothenate (FIG. 4F), dimethyl-guanosine (FIG. 4G) N-acetyl-arginine (FIG. 4H) are higher in the elderly. Metabolite peak areas were divided into 10 bins in each group. Error bars represent means±SD. p-values between age groups are in the range of 0.022 and 0.00039.

FIGS. 5A and 5B are graphs showing that many human blood metabolites exhibit diel constancy. For the great majority of 126 compounds, diel fluctuations in abundance among 4 volunteers were negligible. A. Peak levels of ATP and ergothioneine, like most compounds, hardly changed. The abundance of ergothioneine varies from person to person, however. Furthermore, about 10 compounds, including glycochenodeoxycholate, tetradecanoyl-carnitine, 4-aminobenzoate and caffeine, showed exceptional 24-hr variation. B. Raw abundance data for two example compounds (butyro-betaine and glyceraldehyde-3-phosphate (G-3-P), both enriched in RBCs, determined from 30 individual metabolomes, are shown as dotplots (each dot represents a single individual). Coefficients of variation were 0.35 and 0.99, respectively, for butyro-betaine and G-3-P. Ratios of maximum to minimum abundance are 3.7 and 29, respectively. Peak areas of metabolites were divided into 10 bins in each group. Error bars represent means±SD.

FIGS. 6A to 6C show experimental variability for metabolite measurements which is very small. CV distributions for 126 compounds are shown for (FIG. 6A) CVwi: 3 injections of the same blood sample preparation; (FIG. 6B) CVss: 3 independently prepared samples from the same blood. (FIG. 6C) Coefficients of variation (CV₃₀) for each compound from all 30 blood samples. Most compounds showed negligible CVwi, whereas CVss were more variable and considerably higher for certain metabolites.

FIGS. 7A to 7D show variations of CDP-choline, UDP-glucuronate, phosphocreatine and 4-aminobenzoate. FIGS. 7A and 7B show that two moderately variable metabolites, CDP-choline and UDP-glucuronate, are candidate compounds possibly differing between the two age groups. These compounds are used in biosynthesis and may reflect higher activity levels in younger people. Metabolite peak areas were divided into 10 bins per group. Error bars represent means±SD. FIG. 7C indicates that phosphocreatine shows moderate variation among individuals, but no significant difference between young and elderly. Peak areas were divided into 10 bins per group. Error bars represent means±SD. FIG. 7D shows that 4-aminobenzoate is highly variable among individuals. Peak areas were divided into 10 bins per group. Error bars represent means±SD.

FIGS. 8A to 8E show additional metabolites showing different patterns of abundance between young and elderly groups. NAD+(FIG. 8A), NADP+(FIG. 8B), leucine (FIG. 8C), isoleucine (FIG. 8D) showed higher levels in the youth. N6-acetyl-lysine is higher in elderly people (FIG. 8E). Peak areas of metabolites were divided into 10 bins in each group. Error bars represent means±SD. The range of p-values was 0.0017-0.046.

FIG. 9 shows correlation values for all 14 age-related human blood compounds.

FIGS. 10A to 10F show quantification of blood metabolites from 4 volunteers during prolonged fasting. Experimental procedures employed to study metabolomic changes during human fasting for 58 hr. Four, healthy young volunteers joined the study (FIG. 10A). The right panel shows age, gender, and BMI for each of four volunteers. Blood samples from each person were taken at the indicated timepoints (FIG. 10B). Samples were immediately quenched in 50% methanol at −40° C. Resulting extracts were used for metabolomics analysis. Levels of ATP remained constant in whole blood and plasma during 58 hr of fasting, but increased slightly (1.2˜1.5×) in RBC samples. * denotes statistical significance of p<0.05 (FIG. 10C). Blood glucose levels determined with a glucose tester during fasting. Whole blood glucose levels remained within the normal range (70-80 mg/dL) due to gluconeogenesis during fasting (FIG. 10D). Levels of vital metabolites remained essentially constant during fasting. Profiles of ATP, glutathione disulfide (GSSG), NAD+, and NADP+ in whole blood are shown (FIG. 10E). Scatter plot of 120 metabolites between 10 and 58 hr of fasting. Average whole blood data are shown for the four volunteers. Compounds that displayed minor shifts (within 1.5×˜0.66×) in abundance are placed between two broken lines (FIG. 10F).

FIGS. 11A and 11B show profile of BCAAs and Ophthalmic acid during 58 hr of fasting. BCAAs and Ophthalmic acid (OA) in whole blood or RBC were increased during 58 hr fasting significantly. These are the blood metabolites which are decreased in elderly person.

### DETAILED DESCRIPTION OF EMBODIMENTS

Before one or more embodiments of the present invention are described in detail, it is to be understood that the invention is not limited to the particular methodology, apparatuses, and systems described, as such methodology, apparatuses and systems can, of course, vary. It is also to be understood that the terminology used herein is for the purpose of describing particular embodiments only, and is not intended to limit the scope of the present invention.

Unless defined otherwise or the context clearly dictates otherwise, all technical and scientific terms used herein have the same meaning as commonly understood by one of ordinary skill in the art to which the present disclosure belongs. Any methods and materials similar or equivalent to those described herein can be used in practicing or testing of one or more embodiments of the invention.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

### Definitions

The term “extent of aging” is used herein to refer to the degree of aging or aging index. It is a value indicating whether the speed of aging of the subject is faster (earlier) or slower (later) than the average or the chronological age of the subject. The term “aging” as used herein generally refers to biological aging, as compared to chronological aging that refers to the actual amount of time a subject has been alive. A biological age can be lower or higher than a chronological aging. For example, if the speed of aging of the subject is faster, the biological age is higher than the chronological age.

The term “blood metabolite” is used herein to refer to a low molecular compound involved in biological metabolic activity contained in blood constituents.

It is understood that aspects and embodiments of the invention described herein include “consisting” and/or “consisting essentially of aspects and embodiments.

Other advantages and features according to one or more embodiments of the present invention may be understood from the following specification taken in conjunction with the accompanying drawings.

A Method for Determining the Extent of Aging

According to one or more embodiments of the present invention, the extent of aging is evaluated by using a specific blood metabolite in a subject as an indicator. By measuring the amount of a specific blood metabolite in whole blood, erythrocytes or plasma of the subject, the extent of senescence (aging degree) of the subject can be determined.

Here, the sample used for determining the aging extent of the subject may be at least one kind selected from the group consisting of whole blood, erythrocyte and plasma. It may be preferable to use either whole blood or erythrocyte. It may be more preferable to use any two of whole blood, erythrocyte and plasma. It may be most preferable to use all of whole blood, erythrocyte and plasma as a sample.

As the blood metabolite in one or more embodiments of the present invention, it may be preferable that the compound has a large difference in blood content between the elderly and the young age group. The blood metabolite comprises at least one metabolite selected from the group consisting of Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, Leucine, N2-Acetyl-lysine, Ophthalmic acid, Pantothenate, N6-Acetyl-lysine, NAD+, CDP-choline, Glycerophosphocholine, Histidine, Phenylalanine, Phosphocreatine, Tyrosine, Isoleucine, NADP+, Pentose-phosphate, S-Adenosyl-homocysteine, CDP-ethanolamine, Creatine, CTP, Fructose-6-phosphate, Glycerol-phosphate, Serine, Tryptophan, UDP-glucose, Adenosine, Aspartate, Dimethyl-arginine, Diphospho-glycerate, Glucose-6-phosphate, Glutamate, Glutarate, N-Acetyl-(iso)leucine, and Ketovaline.

Glutathione disulfide (GSSG), UTP, Keto(iso)leucine, 1,5-Anhydroglucitol, Acetyl-carnosine, Carnosine, UDP-acetyl-glucosamine, Leucine, Ophthalmic acid, NAD+, CDP-choline, Glycerophosphocholine, Histidine, Phosphocreatine, Isoleucine, NADP+, Pentose-phosphate, S-Adenosyl-homocysteine, CDP-ethanolamine, CTP, Fructose-6-phosphate, Serine, Tryptophan, UDP-glucose, Adenosine, and Ketovaline are lower in elder. Therefore when the content of these compounds is lower than standard, the extent of aging of the subject is judged to be high.

On the other hand, N-Acetyl-arginine, Citrulline, Dimethyl-guanosine, N2-Acetyl-lysine, Pantothenate, N6-Acetyl-lysine, Phenylalanine, Tyrosine, Creatine, Glycerol-phosphate, Aspartate, Dimethyl-arginine, Diphospho-glycerate, Glucose-6-phosphate, Glutamate, Glutarate and N-Acetyl-(iso)leucine are higher in elder. Therefore when the content of these compounds is higher than standard, the extent of aging of the subject is judged to be high.

In one or more embodiments, the blood metabolite comprises at least one metabolite selected from the group consisting of Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, Leucine, N2-Acetyl-lysine, Ophthalmic acid, Pantothenate, N6-Acetyl-lysine, NAD+, CDP-choline, Glycerophosphocholine, Histidine, Phenylalanine, Phosphocreatine, Tyrosine, Isoleucine, NADP+, Pentose-phosphate, and S-Adenosyl-homocysteine.

In one or more embodiments, the blood metabolite comprises at least one metabolite selected from the group consisting of Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, and Leucine.

In order to obtain a more accurate determination result of aging extent, it may be preferable to analyze plural blood metabolites.

The method for determining the extent of aging according to one or more embodiments of the present invention comprises (i) a step of preparing a sample, (ii) a step of analysis and (iii) a step of determining the extent of aging.

(i) A Step of Preparing a Sample

Metabolomic samples can be prepared as reported previously (NPL 4). All blood samples are drawn in a hospital laboratory to ensure rapid sample preparation. Briefly, venous blood samples for metabolomics analysis are taken into 5 mL heparinized tubes (Terumo). Immediately, 0.1˜1.0 mL blood (4˜60×10⁸ RBC) were quenched in 30˜70% methanol (for example, 50˜60%) of 5˜10 times volume of the blood at −20° C.˜−80° C. (for example, at −40° C.˜−50° C.). This quick quenching step immediately after blood sampling ensured accurate measurement of many labile metabolites. The use of whole blood samples also allowed us to observe cellular metabolite levels that might otherwise have been affected by lengthy cell separation procedures. During Ficoll separation or leukodepletion by filtration, blood cells are exposed to non-physiological conditions for prolonged periods (NPL 4).

The remaining blood sample from each donor is centrifuged at 120 g for 15 min at room temperature to separate plasma and RBCs. After centrifugation, 0.1˜1.0 mL each of separated plasma and RBCs (7-100×10⁸ RBC), are quenched in 30˜70% methanol (for example, 50˜60%) of 5˜10 times volume of the sample at −20° C.˜−80° C. (for example, at −40° C.˜−50° C.). Two internal standards (10 nmol of HEPES and PIPES) are added to each sample. After brief vortexing, samples are transferred to Amicon Ultra 10-kDa cut-off filters (Millipore, Billerica, Mass., USA) to remove proteins and cellular debris. Thus, from each blood sample, three different subsamples, whole blood, RBCs, and plasma, are prepared. The white blood cell content (WBC) is less than 1% of the cellular volume in our preparations (NPL 4). Full metabolomics analysis of WBCs using a Ficoll gradient confirmed that WBCs should not affect our present metabolomics results regarding RBCs. After sample concentration by vacuum evaporation, each sample is re-suspended in 40 μL of 50% acetonitrile, and 1 μL is used for each injection into the LC-MS system.

(ii) A Step of Analysis

The content of blood metabolite in the sample of the subject is analyzed in this step. LC-MS data may be obtained using a Paradigm MS4 HPLC system (Michrom Bioresources, Auburn, Calif., USA) coupled to an LTQ Orbitrap mass spectrometer (Thermo Fisher Scientific, Waltham, Mass., USA), as previously described (NPL 21). Briefly, LC separation is performed on a ZIC-pHILIC column (Merck SeQuant, Umea, Sweden; 150 mm×2.1 mm, 5 μm particle size). The HILIC column is quite useful for separating many hydrophilic blood metabolites, which are previously not assayed by others (NPL 4). Acetonitrile (A) and 10 mM ammonium carbonate buffer, pH 9.3 (B) are used as the mobile phase, with a gradient elution from 80-20% A in 30 min, at a flow rate of 100 μL mL-1. Peak areas of metabolites of interest are measured using MZmine 2 software (87). Detailed data analytical procedures and parameters have been described previously (NPL 21). Metabolomic datasets are deposited in the MetaboLights database (see data availability).

(iii) A Step of Determining the Extent of Aging

We analyze 126 blood compounds confirmed by standards or MS/MS analysis (NPL 4). For each metabolite we choose a singly charged, [M+H]+ or [M−H]−, peak (Table 1). Metabolites are classified into 3 groups (H, M, and L), according to their peak areas. H denotes compounds with high peak areas (>108 AU), M with medium peak areas (108˜107 AU) and L with low peak areas (<107 AU).

As previously reported, some standards at identical molar concentrations, such as AMP and ATP, ionize with different efficiencies, thereby affecting quantification of their peak areas (NPL 21). Thus, in some cases, peak areas could not be reliably converted into actual molar amounts due to different ionization efficiencies of certain compounds between pure sample and metabolite sample mixtures. In this study, however, individually-different relative ratios of peak areas are relevant to obtain CVs so that actual molar concentrations of compounds were not needed.

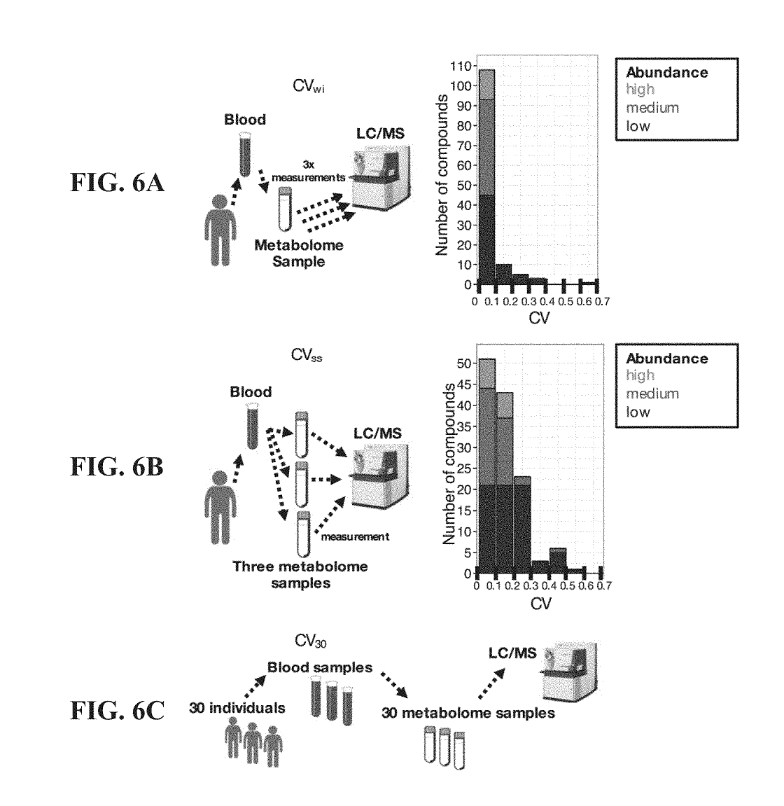

Validation of experimental procedures is performed as follows. First, we evaluate the contribution of sample handling to within-sample variation. The same blood sample preparation is injected 3× into the LC-MS at 80-min intervals (FIG. 6A). We thus obtain within-sample CVs (designated as CVwi), which are less than 0.1 in 107 of 126 compounds (85%). Only 10 compounds show CVwi of 0.1-0.2, while 9 had CVwi>0.2 (Table 1). Most of the variable compounds belong to the low-peak-area (L) group, suggesting that some low-abundance compounds may be labile during LC-MS. However, LC-MS measurements of pure standards for these compounds display much lower CVs (data not shown), implying that their lability results from reactions with other blood compounds or solvent prior to LC-MS measurements.

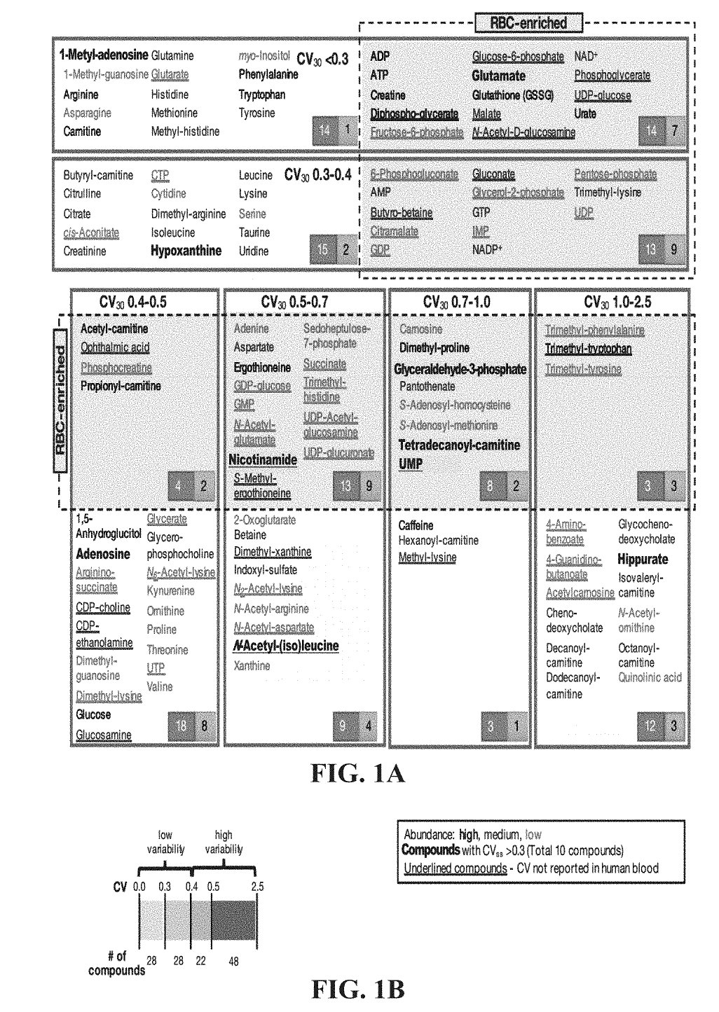

Second, we also examine sample-to-sample variation caused by sample preparation. Three samples are independently prepared from the same blood sample (one person), and CVs thus determined are designated as CVss (FIG. 6B). CVss values of HEPES and PIPES in the blood samples are very small (0.06˜0.08 for HEPES and 0.04˜0.08 for PIPES). The great majority (116/126=92%) of CVss are <0.3 (Table 1). Among 10 compounds with CVss>0.3, 9 compounds belong to the low peak area (L) group. Nicotinamide, a vitamin, with M peak area have CVss=0.47. Three injections of the nicotinamide standard has a CV=0.05, however. Similar situations are observed for 9 other compounds such as UMP and 1-methyl-guanosine, with high CVss values. CVss of these ten compounds in blood may be affected by sample preparation or they may react with other metabolites during sample preparation. Third, we obtain CVs for each blood compound from all 30 volunteers (CV₃₀) (Table 1, FIG. 6C). CV₃₀ were grouped into 6 different value ranges (FIG. 1A).

Raw LC-MS data in mzML format are accessible via the MetaboLights repository (URL: http://www.ebi.ac.uk/metabolights). Data from three injections of the same sample and 3 samples prepared from the same donated blood are available under accession number MTBLS263. Blood samples drawn from four volunteers 4 times within 24 hr are available under accession number MTBLS264. Whole blood metabolomic data from all 30 subjects are available under accession number MTBLS265. Plasma and RBC data from all 30 subjects can be found under MTBLS266 and MTBLS267, respectively.

The method for determining the extent of aging according one or more embodiments of the present invention is not particularly limited as long as it uses the above metabolite as an index. The following method is exemplified as a merely example. The age score (calculated value) can be determined from the data of the aging marker of the subject based on the standard curve made from the plot of the aged marker's quantitative value (peak area) and calendar age. And the extent of aging can be determined by the difference from the calendar age. For example, when dividing the age score of a metabolite by a calendar age and multiplying by 100, the young tendency is judged to be as low as 100 and the older tendency is judged as higher than 100.

A Method for Treating a Subject to Suppress the Extent of Aging

According to one or more embodiments of the present invention, the extent of aging is evaluated by the method described above. When the extent of aging of the subject is judged to be high (or the biological age is high), the extent of aging in the subject may be reduced by fasting. The duration of fasting may be more than 10 hours, more than 20 hours, more than 30 hours, or more than 50 hours. Upper limit can be decided according to the health condition of the subjects, and it is generally 100 hours, 90 hours, 80 hours, 70 hours, or 60 hours. In one or more embodiments, the subjects do not eat or drink anything containing calories (e.g., carbohydrates, proteins, and fats) during the fasting, while carrying out their normal routines. In one or more embodiments, however, a calorie-free diet including a calorie-free food and a calorie-free drink, a hypocaloric diet including a hypocaloric food and a hypocaloric drink, or a fasting mimicking diet may be administered to the subject. The term “calorie-free” used herein may be “zero calorie” or may contain calories at a level that would not affect fasting. Examples of such low calorie foods include, but are not limited to, beans, seeds, vegetable, and processed foods (e.g., indigestible polysaccharide containing foods for calorie restriction). Examples of such drinks include, but are not limited to, water, tea, coffee (e.g., black coffee), sugar-free beverages, and processed beverages (e.g., low fat milk). In one or more embodiments, the fasting includes absolute fast (dry fasting), water fasting, calorie restriction and intermittent fasting. A total calorie intake during the fasting may be 1,000 kcal or less, 750 kcal or less, 500 kcal or less, 250 kcal or less, 100 kcal or less, 50 kcal or less, 40 kcal or less, 30 kcal or less, 20 kcal or less, or 10 kcal or less. The calorie-free diet or the hypocaloric diet may contain calories within the above ranges. In one or more embodiments, the extent of aging in the subjects may be monitored to prepare the blood samples. For example, their blood is sampled on three consecutive weekdays each morning at same time. Their sample preparation and analysis was performed as described above.

Apparatus

One or more embodiments of the present invention provide an apparatus for determining the extent of aging. The apparatus uses the method according to one or more embodiments of the present invention above.

The apparatus for determining the extent of aging according to one or more embodiments of the present invention comprises means for input and means for determining, wherein data of blood metabolites of the subject are input to the means for input, and the extent of aging is determined by comparing the data of the subject with the data of the population. Said method section can be referred for details of the method according to one or more embodiments of the present invention used by the apparatus.

System

One or more embodiments of the present invention provide a system for determining the extent of aging. The extent of aging is determined by the method according to one or more embodiments of the present invention above, or the apparatus according to one or more embodiments of the present invention above. Said method section and the apparatus section can be referred for details of the system according to one or more embodiments of the present invention.

Methods

One or more embodiments of the present invention provide a method of evaluating substances which affect the extent of aging comprising the step of measuring a blood metabolite, wherein the blood metabolite comprises at least one metabolite selected from the group consisting of Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, Leucine, N2-Acetyl-lysine, Ophthalmic acid, Pantothenate, N6-Acetyl-lysine, NAD+, CDP-choline, Glycerophosphocholine, Histidine, Phenylalanine, Phosphocreatine, Tyrosine, Isoleucine, NADP+, Pentose-phosphate, S-Adenosyl-homocysteine, CDP-ethanolamine, Creatine, CTP, Fructose-6-phosphate, Glycerol-phosphate, Serine, Tryptophan, UDP-glucose, Adenosine, Aspartate, Dimethyl-arginine, Diphospho-glycerate, Glucose-6-phosphate, Glutamate, Glutarate, N-Acetyl-(iso)leucine, and Ketovaline. The substances found by this evaluation method can be widely used as anti-aging foods, drinks, supplements, pharmaceuticals, cosmetics and the like. The section of “A method for determining the extent of aging” can be referred for details of the step of measuring a blood metabolite.

Kit

One or more embodiments of the present invention provide a kit for determining the extent of aging by using the methods according to one or more embodiments of the present invention, comprising blood collection tubes and blood metabolite compounds as detection standard. The kit according to one or more embodiments of the present invention may comprise any constituent elements besides the blood collection tube and the like. The blood metabolite compounds as detection standard can be selected from the group consisting Glutathione disulfide (GSSG), UTP, Keto(iso)leucine, N-Acetyl-arginine, 1,5-Anhydroglucitol, Acetyl-carnosine, Citrulline, Dimethyl-guanosine, Carnosine, UDP-acetyl-glucosamine, Leucine, N2-Acetyl-lysine, Ophthalmic acid, Pantothenate, N6-Acetyl-lysine, NAD+, CDP-choline, Glycerophosphocholine, Histidine, Phenylalanine, Phosphocreatine, Tyrosine, Isoleucine, NADP+, Pentose-phosphate, S-Adenosyl-homocysteine, CDP-ethanolamine, Creatine, CTP, Fructose-6-phosphate, Glycerol-phosphate, Serine, Tryptophan, UDP-glucose, Adenosine, Aspartate, Dimethyl-arginine, Diphospho-glycerate, Glucose-6-phosphate, Glutamate, Glutarate, N-Acetyl-(iso)leucine, and Ketovaline.

Metabolomics of RBCs.

Untargeted metabolomics of human blood by LC-MS (NPL 4) was performed to evaluate individual variation among healthy subjects, using the coefficient of variation (CV). Our technique, including rapid quenching of samples, whole blood analysis without centrifugation and use of an HILIC column, partly explain why we succeeded in identifying hitherto unreported CVs for many metabolites. We emphasized the importance of RBC metabolomics. This is not simply due to the scarcity of such studies, but because RBCs serve such a crucial function. For example, abundant anti-oxidants in blood, such as glutathione, are exclusively enriched in RBCs over 1,000×. In addition, we show ophthalmic acid and carnosine, both related to anti-oxidants, are RBC-enriched and their abundance seems age-dependent. RBCs thus seem to play the central role in anti-oxidation in blood. Many cellular compounds, such as sugar phosphates, nucleotides, and nucleotide-sugar derivatives for energy production are enriched in RBCs. Since half the blood volume is occupied by RBCs, RBC metabolomics may be as important as that of plasma to understand the diverse functions of human blood.

Blood Metabolites with High CVs as Personal Markers.

We identified 48 metabolites showing moderate to very high CV₃₀ (0.5˜2.3). To our knowledge, CVs of 22 of these compounds have not been previously reported. For the most part, these compounds do not fluctuate on a diel basis; thus we suppose that individual variability may reflect (epi)genetic differences or chronic states. To fully explore their potential as personal markers, further investigation of physiological roles of these compounds is required. Compounds with low CVs may support physiological homeostasis in vivo. Indeed, anomalous glutathione levels are reported in many diseases, such as Parkinson's disease, HIV, liver disease, and cystic fibrosis, as well as aging. A number of diseases are reportedly relevant to degradation pathways for leucine, valine, and isoleucine. Thus, low CV compounds might be good candidates as health markers.

Increases of Certain Age-Related Compounds in Blood of the Elderly.

Our metabolomic comparisons of human blood, including RBCs, between young and elderly subjects revealed 14 age-related compounds. Six of them are RBC-enriched. Our results regarding CV₃₀ for three of the 14 compounds (1,5-anhydroglucitol, pantothenate, citrulline) are confirmatory to the previous study: 1,5-anhydroglucitol is higher in young people (1), while pantothenate and citrulline are more abundant in healthy elderly persons (2, 3). The design of our approach might help us to identify these novel aspects with statistical significance, even though the population for the study was not large (N=30). Exclusion of middle-aged people (40˜70 years old) from the study gave us clearer age-difference between two groups. Samples were also collectively analyzed at one time for the accurate measurement.

Eight of the remaining novel age-related 11 compounds are lower in elderly subjects. Our results suggest that the blood of elderly subjects shows reduced levels of some compounds related to anti-oxidants (ophthalmic acid, carnosine etc.) and redox metabolites (NAD+, NADP+), as well as compounds that support muscle maintenance and reinforcement (leucine, isoleucine).

In contrast, three plasma-enriched compounds (N-acetyl-arginine, dimethyl-guanosine and N6-acetyl-lysine) increase in the elderly. N-acetyl-arginine and citrulline, the by-products of the urea cycle, might increase due to impaired efficiency of this cycle. Indeed, deficiencies of urea cycle enzymes are known to cause the accumulations of these compounds (4, 5). Dimethyl-guanosine is known to increase in the plasma of uremic patients (6). These results suggest that gradual, progressive decay of liver or renal function may be typical among elderly people generally, resulting in a gradual rise in these blood metabolites.

Certain Compounds Supporting Vigorous Activity Decline in Elderly.

It is also noteworthy that several age-related compounds, including carnosine, are identified in RBC analysis. Carnosine (beta-alanyl-L-histidine), a possible scavenger of oxidants, is highly-concentrated in muscle and brain. Our data demonstrate that carnosine is a highly variable metabolite enriched in RBC. These findings allow us to reconsider the physiological role of RBCs in blood. RBCs may also serve to transport carnosine and other metabolites to distant tissues. Consistently acetyl-carnosine, which is resistant to degradation, is plasma-enriched. The RBC/plasma ratios among 30 subjects are 10.8 (carnosine) and 0.13 (acetyl-carnosine). Carnosine is clearly RBC-enriched while acetyl-carnosine is clearly a plasma compound. Our study demonstrated that both compounds decline in the elderly. Further study to elucidate the role of carnosine in RBCs is of considerable interest.

Anti-Oxidants, and Compounds Related to Energy and Cellular Maintenance in Blood.

Compounds required for vigorous activity during youth may decline in the elderly. Ophthalmic acid is related to glutathione, and both are generated by the same biosynthetic enzymes. Hence ophthalmic acid is thought to be related to anti-oxidant; it also decreases in the elderly. The level of UDP-acetyl-glucosamine was 2-fold higher in young than in elderly subjects. This compound is required for cell signaling during proteoglycan and glycolipid synthesis and for the formation of nuclear pores. These functions are compatible with the hypothesis that synthesis of anti-oxidants and cellular maintenance compounds declines with age. Consistently, leucine, isoleucine, NAD+, and NADP+, which are more abundant in youth, may suggest that these compounds are more vigorously consumed in the body, particularly in muscle, when physical activity is higher. It is unclear whether lower levels of these compounds result in diminished muscle and possibly brain activity, or whether they reflect reduced activity. Scavengers of oxidants may be required to restore energy-related biochemical reactions in RBCs.

Future Prospects of Human Metabolomics.

It is noteworthy that 38 of these 43 age-related compounds (except for 1,5-anhydroglucitol, carnosine, creatine, phosphocreatine and acetyl-carnosine) are also present in fission yeast. In the near future, genetics of these compounds in fission yeast and other organisms may be helpful to dissect their physiological and cytological significance. If so, the present analysis of RBCs, plasma, and whole blood will support the development of human metabolomics. It could be considered that 43 blood metabolites found in one or more embodiments of the present invention are correlated with aging related diseases. Based on the blood levels of these metabolites as indicators, it may be possible to determine risk of disease, status of disease, susceptibility to disease, etc. Following diseases can be exemplified as said diseases. Lifestyle-related disease (such as atherosclerosis, hypertension, type 2 diabetes, miletus, menopause, osteoporosis, cancer); Neurological disorder (such as brain infarction, Altzheimer disease, dementia, Parkinson Syndrome); Eye disease (such as cataract, glaucoma, age-related macular degeneration, presbyopia, dry eye); Otorhinolaryngologic disease (such as hearing disturbance, chronic thyroiditis, xerostomia); Hematological disorder (such as malignant lymphoma, leukemia, anemia); Heart disease (such as ischemic heart disease, myocardial infarction, heart failure, angina pectoris, acute coronary syndrome); Pulmonary disease (such as COPD(Chronic obstructive pulmonary Disease), lung fibrosis); Digestive disease (such as atrophic gastritis, liver cirrhosis, fatty liver, liver dysfunction); Kidney & urological disease (such as urine incontinence, late onset hypogonadism syndrome, chornic renal failure, prostate hypertrophy), Musculoskeletal disease (such as arthritis, locomotive syndrome, sarcopenia, lumbago, joint pain, frailty), poor nutrition, progeria, Werner syndrome and the like.

### Examples

The summary of the Examples is as follows. We report blood metabolites of 30 individuals in a study having three distinct facets. We analyzed 126 blood metabolites which are confirmed by standards or MS/MS analysis (NPL 4). For each metabolite we chose a singly charged, [M+H]+ or [M−H]−, peak (Table 1). We collected samples from RBCs, plasma, and whole blood for metabolomics analysis. Combining the present quantitative data with previous analysis of RBCs and white blood cells (WBCs) carefully separated from RBCs (NPL 4), we now have ample knowledge of metabolites enriched in RBCs. RBC-enriched metabolites may reflect health status or environmental stresses differently than do metabolites of plasma.

Second, to quantify individual variation, we employed a simple parameter designated the coefficient of variation (CV), for each blood compound. The CV is the ratio of the standard deviation (SD) of metabolite abundance (peak areas from LC-MS) divided by the mean. For stable and relatively invariant metabolites, SDs and CVs are low or negligible, while CVs of variable metabolites may prove useful in the evaluation of metabolite variation among individuals. RBC and plasma metabolites from 30 volunteers were analyzed using LC-MS (NPL 4, 21). HEPES and PIPES were spiked into all blood samples as internal standards. CVs of any compounds significantly larger than those of HEPES and PIPES were candidates to be analyzed for individual metabolite variation.

Thirdly, comparisons of blood metabolomes between young and elderly volunteers were performed with emphasis on RBC metabolites that have seldom been considered as targets of age analysis. We were able to identify a total of 43 metabolites statistically relevant to aging. Four of them were previously reported, but we believe that others hitherto have not been. We discuss our findings in regard to human aging.

TABLE 1
List of 126 identified blood metabolites^{a}

| | | | | | | | |
|---|---|---|---|---|---|---|---|
| | | Reported | | | | | p-value |
| | | concentration | | | | | between |
| | RBC | in plasma or | | | | CV | age |
| Category/Compound | enriched | blood (μM) | Abundance | CVₛₛ | CV₃₀ | reported | groups |

Nucleotides (7/11)

| | | | | | | | |
|---|---|---|---|---|---|---|---|
| ADP | X | 48 ± 13 | H-M | 0.05 | 0.27 | X | 0.64 |
| AMP | X | 6.2 ± 3.1 | H-M | 0.07 | 0.33 | X | 0.71 |
| ATP | X | 433 ± 73 | H-M | 0.07 | 0.17 | X | 0.31 |
| CTP | | | L | 0.1 | 0.33 | | 0.078 |
| GDP | X | 15 ± 2 | L | 0.09 | 0.38 | | 0.075 |
| GTP | X | 25 ± 6 | H-M | 0.05 | 0.32 | X | 0.58 |
| GMP | X | 0 | L | 0.24 | 0.57 | | 0.95 |
| IMP | X | 14-100 | L | 0.06 | 0.38 | | 0.55 |
| UDP | X | 0 | L | 0.11 | 0.31 | | 0.79 |
| UMP | X | 0 | L | 0.58 | 0.88 | | 0.86 |
| UTP | | | L | 0.25 | 0.44 | | 0.46 |

Nucleosides, nucleobases and derivatives (1/12)

| | | | | | | | |
|---|---|---|---|---|---|---|---|
| 1-Methyl-adenosine | | 0.06 ± 0.006 | H-M | 0.45 | 0.29 | X | 0.63 |
| 1-Methyl-guanosine ** | | 0.046 ± 0.019 | L | 0.23 | 0.25 | X | 0.25 |
| Adenine | X | 0.3 ± 0.28 | L | 0.2 | 0.53 | X | 0.71 |
| Adenosine | | 0.5 ± 0.1 | L | 0.38 | 0.49 | X | 0.052 |
| Caffeine | | 0-35 | H-M | 0.29 | 0.92 | X | 0.43 |
| Cytidine | | 0.25 ± 0.19 | L | 0.08 | 0.33 | X | 0.079 |
| Dimethyl-guanosine | | 0.029 ± 0.005 | L | 0.26 | 0.46 | X | 0.0081 |
| Dimethyl-xanthine | | 0-8 | H-M | 0.25 | 0.61 | | 0.078 |
| Hypoxanthine ** | | 0.38 ± 0.18 | H-M | 0.32 | 0.35 | X | 0.98 |
| Urate | X | 336 ± 94 | H-M | 0.07 | 0.28 | X | 0.15 |
| Uridine * | | 3.12 ± 1.31 | H-M | 0.14 | 0.34 | X | 0.84 |
| Xanthine * | | 1.27 ± 0.78 | L | 0.03 | 0.56 | X | 0.87 |

Vitamins, Coenzymes (2/5)

| | | | | | | | |
|---|---|---|---|---|---|---|---|
| 4-Aminobenzoate | | <1 ng/ml | L | 0.03 | 2.18 | | 0.32 |
| NAD⁺ | X | 23.3 ± 6.9 | H-M | 0.08 | 0.3 | X | 0.046 |
| NADP⁺ | X | 19.6 ± 6.6 | H-M | 0.11 | 0.36 | X | 0.022 |
| Nicotinamide | X | 0.45 | H-M | 0.47 | 0.56 | | 0.38 |
| Pantothenate | X | 2.2 ± 1.02 | H-M | 0.2 | 0.82 | X | 0.022 |

Nucleotide-sugar derivatives (4/4)

| | | | | | |
|---|---|---|---|---|---|
| GDP-glucose | X | L | 0.09 | 0.53 | 0.26 |
| UDP-acetyl-glucosamine | X | L | 0.14 | 0.64 | 0.0073 |
| UDP-glucose | X | H-M | 0.18 | 0.24 | 0.088 |
| UDP-glucuronate | X | L | 0.24 | 0.63 | 0.12 |

Sugar phosphates (8/9)

| | | | | | | | |
|---|---|---|---|---|---|---|---|
| 6-Phosphogluconate | X | | L | 0.25 | 0.3 | | 0.49 |
| Diphospho-glycerate | X | 4500 | H-M | 0.17 | 0.24 | | 0.77 |
| Fructose-6-phosphate | X | 16 | L | 0.21 | 0.24 | | 0.61 |
| Glucose-6-phosphate | X | 38 | H-M | 0.19 | 0.29 | | 0.94 |
| Glyceraldehyde-3- | X | 6.7 ± 1.0 | L | 0.49 | 0.99 | | 0.86 |
| phosphate ** | | | | | | | |
| Glycerol-2-phosphate ** | X | | L | 0.16 | 0.31 | | 0.32 |
| Pentose phosphate | X | 13.2 ± 4.8 | L | 0.28 | 0.34 | | 0.049 |
| Phosphoglycerate | X | 58 ± 14 | H-M | 0.24 | 0.29 | | 0.12 |
| Sedoheptulose-7- | X | 0.89 ± 0.41 | L | 0.28 | 0.52 | X | 0.82 |
| phosphate | | | | | | | |

Sugars and derivatives (3/6)

| | | | | | | | |
|---|---|---|---|---|---|---|---|
| 1,5-Anhydroglucitol | | 120 ± 39 | H-M | 0.08 | 0.46 | X | 0.0004 |
| Gluconate | X | <5 | H-M | 0.16 | 0.33 | | 0.32 |
| Glucosamine | | 0.23-0.68 | H-M | 0.19 | 0.47 | | 0.21 |
| Glucose | | 4700-6100 | H-M | 0.15 | 0.4 | X | 0.54 |
| myo-inositol * | | 21-49 | L | 0.27 | 0.24 | X | 0.31 |
| N-Acetyl-D-glucosamine | X | | H-M | 0.09 | 0.26 | | 0.82 |

Organic acids (6/10)

| | | | | | | | |
|---|---|---|---|---|---|---|---|
| 2-Oxoglutarate | | 8.6 ± 2.6 | L | 0.19 | 0.54 | X | 0.63 |
| Chenodeoxycholate | | 0.85 ± 0.88 | H-M | 0.05 | 1.33 | X | 0.77 |
| Glycochenodeoxycholate | | 0.06 ± 0.01 | H-M | 0.04 | 1.2 | X | 0.93 |
| cis-Aconitate | | | L | 0.14 | 0.35 | | 0.11 |
| Citramalate | X | | L | 0.15 | 0.36 | | 0.82 |
| Citrate | | 79 ± 27 | H-M | 0.08 | 0.31 | X | 0.59 |
| Gluterate | | 0.0-1.8 | L | 0.16 | 0.29 | | 0.65 |
| Glycerate | | 0-24 | L | 0.02 | 0.43 | | 0.39 |
| Malate | X | 0-21 | H-M | 0.17 | 0.2 | | 0.76 |
| Succinate | X | 8.8 ± 2.7 | L | 0.25 | 0.6 | | 0.49 |

Standard amino acids (0/17)

| | | | | | | | |
|---|---|---|---|---|---|---|---|
| Arginine | | 80 ± 20 | H-M | 0.04 | 0.29 | X | 0.64 |
| Asparagine | | 41 ± 10 | L | 0.17 | 0.23 | X | 0.11 |
| Aspartate | X | 3 ± 1,400 ± 120 | H-M | 0.27 | 0.5 | X | 0.62 |
| Glutamate ** | X | 25 ± 15, | H-M | 0.44 | 0.28 | X | 0.29 |
| | | 1110 ± 360 | | | | | |
| Glutamine | | 586 ± 84 | H-M | 0.16 | 0.2 | X | 0.14 |
| Histidine | | 82 ± 10 | H-M | 0.12 | 0.19 | X | 0.06 |
| Isoleucine | | 62 ± 14 | H-M | 0.18 | 0.32 | X | 0.012 |
| Leucine | | 123 ± 25 | H-M | 0.13 | 0.31 | X | 0.002 |
| Lysine | | 188 ± 32 | H-M | 0.24 | 0.39 | X | 0.87 |
| Methionine | | 25 ± 4 | H-M | 0.19 | 0.28 | X | 0.17 |
| Phenylalanine | | 57 ± 9 | H-M | 0.01 | 0.17 | X | 0.041 |
| Proline | | 168 ± 60 | L | 0.19 | 0.42 | X | 0.47 |
| Serine | | 114 ± 19 | L | 0.27 | 0.33 | X | 0.2 |
| Threonine | | 140 ± 33 | L | 0.17 | 0.42 | X | 0.63 |
| Tryptophan | | 44 ± 7 | H-M | 0.08 | 0.24 | X | 0.22 |
| Tyrosine | | 59 ± 12 | H-M | 0.06 | 0.27 | X | 0.051 |
| Valine | | 233 ± 43 | L | 0.2 | 0.48 | X | 0.95 |

Methylated amino acids (8/13)

| | | | | | | | |
|---|---|---|---|---|---|---|---|
| Betaine | | 47 ± 18 | H-M | 0.06 | 0.51 | X | 0.26 |
| Butyro-betaine | X | 0.76 | H-M | 0.12 | 0.35 | | 0.19 |
| Dimethyl-arginine | | 0.66 ± 0.19 | H-M | 0.05 | 0.31 | X | 0.079 |
| Dimethyl-lysine | | | L | 0.04 | 0.44 | | 0.078 |
| Dimethyl-proline | X | 7 ± 10.8 | H-M | 0.03 | 0.79 | X | 0.68 |
| (stachydrine) | | | | | | | |
| Methyl-histidine | | 16.5 ± 10.1 | H-M | 0.04 | 0.3 | X | 0.45 |
| Methyl-lysine | | 7 ± 6 | H-M | 0.03 | 0.73 | | 0.66 |
| S-methyl- | X | | H-M | 0.05 | 0.63 | | 0.36 |
| ergothioneine | | | | | | | |
| Trimethyl-histidine | X | | L | 0.17 | 0.57 | | 0.21 |
| (hercynine) | | | | | | | |
| Trimethyl-lysine | X | 0.56 ± 017 | H-M | 0.09 | 0.38 | X | 0.38 |
| Trimethyl- | X | | L | 0.11 | 1.18 | | 0.9 |
| phenylalanine | | | | | | | |
| Trimethyl-tryptophan | X | | H-M | 0.03 | 1.67 | | 0.96 |
| (hypaphorine) | | | | | | | |
| Trimethyl-tyrosine ** | X | | L | 0.25 | 2.5 | | 0.21 |

Acetylated amino acid (5/7)

| | | | | | | | |
|---|---|---|---|---|---|---|---|
| N-Acetyl-(iso)leucine | | | L | 0.36 | 0.63 | | 0.056 |
| N-Acetyl-arginine | | 1.25 ± 0.28 | L | 0.13 | 0.62 | X | 0.0004 |
| N-Acetyl-aspartate | | <0.35 | L | 0.11 | 0.58 | | 0.18 |
| N-Acetyl-glutamate | X | | L | 0.07 | 0.57 | X | 0.16 |
| N-Acetyl-ornithine * | | 1.1 ± 0.4 | L | 0.05 | 1.17 | | 0.98 |
| N₂-Acetyl-lysine | | | L | 0.07 | 0.5 | | 0.033 |
| N₆-Acetyl-lysine * | | | L | 0.13 | 0.43 | | 0.012 |

Other amino acid derivatives (4/16)

| | | | | | | | |
|---|---|---|---|---|---|---|---|
| 4-Guanidinobutanoate * | | <0.013-0.055 | L | 0.11 | 2.05 | | 0.19 |
| Acetyl-carnosine | | | L | 0.09 | 1.07 | | 0.001 |
| Arginino-succinate ** | | | L | 0.27 | 0.4 | | 0.61 |
| Carnosine * | X | 6.5 ± 2.8 | L | 0.19 | 0.79 | X | 0.003 |
| Citrulline | | 40 ± 10 | H-M | 0.06 | 0.3 | X | 0.001 |
| Creatine | X | 30.1 ± 12.3 | H-M | 0.12 | 0.29 | X | 0.58 |
| Creatinine | | 82.6 ± 26.2 | H-M | 0.1 | 0.35 | X | 0.23 |
| Hippurate | | 4.28 ± 2.61 | H-M | 0.42 | 1.01 | X | 0.28 |
| Indoxyl-sulfate | | 2.5 ± 1.4 | H-M | 0.15 | 0.59 | X | 0.071 |
| Kynurenine | | 1.35 ± 0.26 | L | 0.06 | 0.48 | X | 0.91 |
| Ornithine | | 55 ± 16 | L | 0.26 | 0.48 | X | 0.32 |
| Phosphocreatine | X | | L | 0.07 | 0.48 | | 0.65 |
| Quinolinic acid | | 0.47 ± 0.22 | L | 0.3 | 1.18 | X | 0.23 |
| S-Adenosyl- | X | 0.46 ± 0.02 | L | 0.08 | 0.83 | X | 0.044 |
| homocysteine * | | | | | | | |
| S-Adenosyl- | X | 0.68 ± 0.03 | L | 0.15 | 0.88 | X | 0.085 |
| methionine ** | | | | | | | |
| Taurine | | 55 ± 13 | H-M | 0.1 | 0.37 | X | 0.79 |

Carnitines (0/10)

| | | | | | | | |
|---|---|---|---|---|---|---|---|
| Acetyl-carnitine | X | 10.2 ± 2.2 | H-M | 0.05 | 0.41 | X | 0.78 |
| Butyryl-carnitine | | 0.267 ± 0.077 | H-M | 0.02 | 0.37 | X | 0.19 |
| Carnitine | | 30 ± 11 | H-M | 0.07 | 0.2 | X | 0.41 |
| Decanoyl-carnitine | | 0.141 ± 0.053 | H-M | 0.12 | 1.11 | X | 0.47 |
| Dodecanoyl-carnitine | | 0.052 ± 0.024 | H-M | 0.14 | 1.11 | X | 0.49 |
| Hexanoyl-carnitine | | 0.080 ± 0.035 | H-M | 0.04 | 0.76 | X | 0.8 |
| Isovaleryl-carnitine | | 0.138 ± 0.059 | H-M | 0.08 | 1.98 | X | 0.25 |
| Octanoyl-carnitine | | 0.121 ± 0.053 | H-M | 0.25 | 1.02 | X | 0.61 |
| Propionyl-carnitine | X | 0.400 ± 0.124 | H-M | 0.02 | 0.41 | X | 0.68 |
| Tetradecanoyl- | X | 0.024 ± 0.006 | L | 0.45 | 0.47 | X | 0.15 |
| Carnitine ** | | | | | | | |

Choline derivative (2/3)

| | | | | | | |
|---|---|---|---|---|---|---|
| CDP-choline | <3 μM | H-M | 0.07 | 0.41 | | 0.088 |
| CDP-ethanolamine * | <3 μM | L | 0.18 | 0.43 | | 0.077 |
| Glycerophosphocholine | 32 ± 3 | H-M | 0.08 | 0.47 | X | 0.066 |

Antioxidant (1/3)

| | | | | | | | |
|---|---|---|---|---|---|---|---|
| Ergothioneine | X | 56 ± 47 | H-M | 0.12 | 0.63 | X | 0.2 |
| Glutathione disulfide | X | 1.69 ± 0.38, | H-M | 0.19 | 0.18 | X | 0.21 |
| (GSSG) | | 3210 ± 1500 | | | | | |
| Ophthalmic acid * | X | 0.01-0.03, | H-M | 0.12 | 0.43 | | 0.0087 |
| | | 11.8-16.4 | | | | | |

Standards

| | | | | |
|---|---|---|---|---|
| HEPES[M + H]⁺ | H-M | 0.06 | 0.29 | 0.761 |
| HEPES[M − H]⁻ | H-M | 0.08 | 0.21 | 0.02 |
| PIPES[M + H]⁺ | H-M | 0.04 | 0.14 | 0.061 |
| PIPES[M − H]⁻ | H-M | 0.08 | 0.23 | 0.113 |

^{a}126 compounds and 2 standards (HEPES, PIPES) detected by LC-MS are shown. Compounds with a single asterisk displayed within-sample variation (CV_{wi}) = 0.1-0.2, while no asterisk indicates a CV_{wi} less than 0.1. Those with two asterisks had CV_{wi} > 0.2. Compound specifically enriched in RBCs, as opposed to plasma, are indicated (X). Compound concentrations previously reported in the literature (X) are shown (references in Dataset S1). Compound abundance (peak area) is indicated (H-M—high-medium, L—low). Coefficients of variation among three preparations from the same blood sample were denominated (CVₛₛ). Ten compounds had CVₛₛ values >0.3 and 23 compounds had CVₛₛ values >0.2 (highlighted in bold). Coefficients of variation for each compound among 30 subjects (CV₃₀) are summarized in FIG. 1 and correspond roughly to other values in the literature. Compounds for which CVs have been reported previously are indicated (X). CV₃₀ values >0.4 highlighted in bold. Levels of compounds between young and elderly subjects were considered to differ significantly at p < 0.05. Raw data are deposited in the MetaboLights database (see data availability).

Ethics Statement

Written, informed consent was obtained from all donors, in accordance with the Declaration of Helsinki. All experiments were performed in compliance with relevant Japanese laws and institutional guidelines. All protocols were approved by the Ethical Committee on Human Research of Kyoto University Hospital and by the Human Subjects Research Review Committee of the Okinawa Institute of Science and Technology Graduate University (OIST).

Human Subject Characteristics and Blood Metabolomics Analysis

30 healthy male and female volunteers participated in this study (Table 2). Metabolomic samples were prepared as reported previously (NPL 4). Detailed procedures of LC-MS measurements and determination of CVs for each metabolite can be found in SI

Blood samples for metabolomics analysis and clinical blood parameters were taken in the morning and subjects were asked not to eat breakfast to ensure at least 12 hr of fasting.

TABLE 2
Human subject characteristics.

| | Youth (n = 15) | Elderly (n = 15) | All (n = 30) |
|---|---|---|---|
| Age (median, IQR) | 28.2 (26.1, 31.2) | 80.6 (76.0, 84.3) | 53.5 (28.2, 80.6) |
| Gender (male:female) | 10; 5 | 4; 11 | 14; 16 |
| Hb, g/dL | 15.3 (14.2, 16.0) | 13.5 (12.8, 14.1) | 14.1 (13.3, 15.6) |
| Hemoglobin A1c (median, IQR), % | 4.7 (4.5, 4.9) | 5.2 (4.9, 5.4) | 4.9 (4.7, 5.2) |
| WBC (median, IQR), ×10⁹/L | 5.8 (5.1, 6.3) | 4.9 (4.2, 6.4) | 5.5 (4.5, 6.4) |
| RBC (median, IQR), ×10¹²/L | 5.1 (4.8, 5.2) | 4.2 (4.0, 4.6) | 4.7 (4.2, 5.2) |
| MCV (median, IQR), fL | 92.0 (89.5, 94.5) | 97.8 (92.1, 102.4) | 94.3 (90.5, 97.3) |
| Platelets (median, IQR), ×10¹¹/L | 20.8 (20.0, 27.0) | 18.3 (15.1, 23.5) | 20.6 (16.4, 26.2) |
| HT hematocrite (median, IQR), % | 45.9 (44.0, 47.4) | 41.9 (41.1, 44.5) | 44.0 (41.7, 46.7) |
| Glucose (median, IQR), mg/dL | 88.5 (86.3, 92.8) | 96.0 (91.5, 100.3) | 92.5 (87.0, 95.5) |
| Creatinine (median, IQR), mg/dL | 0.8 (0.7, 0.8) | 0.7 (0.5, 0.8) | 0.7 (0.6, 0.8) |

Blood Sample Preparation for Metabolomics Analysis

Metabolomic samples were prepared as reported previously (NPL 4). All blood samples were drawn in a hospital laboratory to ensure rapid sample preparation. Briefly, venous blood samples for metabolomics analysis were taken into 5 mL heparinized tubes (Terumo). Immediately, 0.2 mL blood (8−12×10⁸ RBC) were quenched in 1.8 ml 55% methanol at −40° C. This quick quenching step immediately after blood sampling ensured accurate measurement of many labile metabolites. The use of whole blood samples also allowed us to observe cellular metabolite levels that might otherwise have been affected by lengthy cell separation procedures. During Ficoll separation or leukodepletion by filtration, blood cells are exposed to non-physiological conditions for prolonged periods (NPL 4).

The remaining blood sample from each donor was centrifuged at 120 g for 15 min at room temperature to separate plasma and RBCs. After centrifugation, 0.2 mL each of separated plasma and RBCs (14−20×10⁸ RBC), were quenched in 1.8 mL 55% methanol at −40° C. Two internal standards (10 nmol of HEPES and PIPES) were added to each sample. After briefvortexing, samples were transferred to Amicon Ultra 10-kDa cut-off filters (Millipore, Billerica, Mass., USA) to remove proteins and cellular debris. Thus, from each blood sample, three different subsamples, whole blood, RBCs, and plasma, were prepared. The white blood cell content (WBC) is less than 1% of the cellular volume in our preparations (NPL 4). Full metabolomics analysis of WBCs using a Ficoll gradient confirmed that WBCs should not affect our present metabolomics results regarding RBCs. After sample concentration by vacuum evaporation, each sample was re-suspended in 40 μL of 50% acetonitrile, and 1 μL was used for each injection into the LC-MS system.

LC-MS Analysis

LC-MS data were obtained using a Paradigm MS4 HPLC system (Michrom Bioresources, Auburn, Calif., USA) coupled to an LTQ Orbitrap mass spectrometer (Thermo Fisher Scientific, Waltham, Mass., USA), as previously described (NPL 21). Briefly, LC separation was performed on a ZIC-pHILIC column (Merck SeQuant, Umea, Sweden; 150 mm×2.1 mm, 5 μm particle size). The HILIC column is quite useful for separating many hydrophilic blood metabolites, which were previously not assayed by others (NPL 4). Acetonitrile (A) and 10 mM ammonium carbonate buffer, pH 9.3 (B) were used as the mobile phase, with a gradient elution from 80-20% A in 30 min, at a flow rate of 100 μL mL-1. Peak areas of metabolites of interest were measured using MZmine 2 software. Detailed data analytical procedures and parameters have been described previously (NPL 21). Metabolomic datasets are deposited in the MetaboLights database (see data availability).

CVs of Blood Metabolites Analyzed by LC-MS

We analyzed 126 blood compounds confirmed by standards or MS/MS analysis (NPL 4). For each metabolite we chose a singly charged, [M+H]+ or [M−H]−, peak (Table 1). Metabolites were classified into 3 groups (H, M, and L), according to their peak areas. H denotes compounds with high peak areas (>10⁸ AU), M with medium peak areas (10⁸˜10⁷ AU) and L with low peak areas (<107 AU).

As previously reported, some standards at identical molar concentrations, such as AMP and ATP, ionize with different efficiencies, thereby affecting quantification of their peak areas (NPL 21). Thus, in some cases, peak areas could not be reliably converted into actual molar amounts due to different ionization efficiencies of certain compounds between pure sample and metabolite sample mixtures. In this study, however, individually-different relative ratios of peak areas are relevant to obtain CVs so that actual molar concentrations of compounds were not needed.

Determination of CVs for Each Metabolite

Validation of experimental procedures was performed as follows. First, we evaluated the contribution of sample handling to within-sample variation. The same blood sample preparation was injected 3× into the LC-MS at 80-min intervals (FIG. 6A). We thus obtained within-sample CVs (designated as CVwi), which were less than 0.1 in 107 of 126 compounds (85%). Only 10 compounds showed CVwi of 0.1-0.2, while 9 had CVwi>0.2 (Table 1). Most of the variable compounds belonged to the low-peak-area (L) group, suggesting that some low-abundance compounds may be labile during LC-MS. However, LC-MS measurements of pure standards for these compounds displayed much lower CVs (data not shown), implying that their lability results from reactions with other blood compounds or solvent prior to LC-MS measurements.

Second, we also examined sample-to-sample variation caused by sample preparation. Three samples were independently prepared from the same blood sample (one person), and CVs thus determined were designated as CVss (FIG. 6B). CVss values of HEPES and PIPES in the blood samples were very small (0.06˜0.08 for HEPES and 0.04˜0.08 for PIPES). The great majority (116/126=92%) of CVss were <0.3 (Table 1). Among 10 compounds with CVss>0.3, 9 compounds belonged to the low peak area (L) group. Nicotinamide, a vitamin, with M peak area had CVss=0.47. Three injections of the nicotinamide standard had a CV=0.05, however. Similar situations were observed for 9 other compounds such as UMP and 1-methyl-guanosine, with high CVss values. CVss of these ten compounds in blood may be affected by sample preparation or they may react with other metabolites during sample preparation. Third, we obtained CVs for each blood compound from all 30 volunteers (CV₃₀) (Table 1, FIG. 6C). CV₃₀ were grouped into 6 different value ranges (FIG. 1A).

Data Availability

Raw LC-MS data in mzML format are accessible via the MetaboLights repository (URL: http://www.ebi.ac.uk/metabolights). Data from three injections of the same sample and 3 samples prepared from the same donated blood are available under accession number MTBLS263. Blood samples drawn from four volunteers 4 times within 24 hr are available under accession number MTBLS264. Whole blood metabolomic data from all 30 subjects are available under accession number MTBLS265. Plasma and RBC data from all 30 subjects can be found under MTBLS266 and MTBLS267, respectively.

Diel Variation of Blood Metabolites; Many Metabolite Levels are Constant on a Daily Basis.

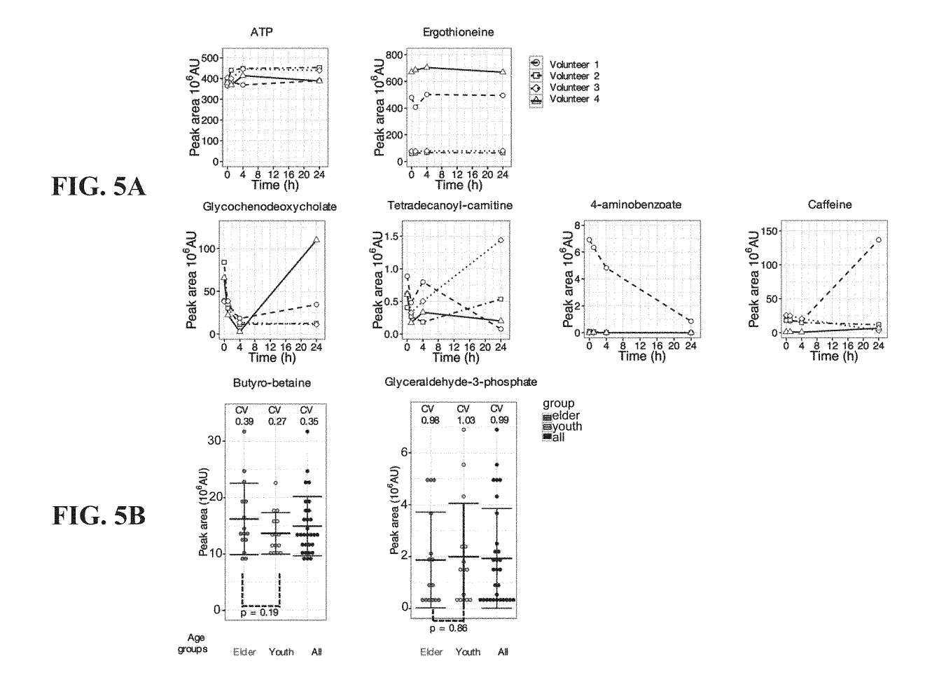

We first investigated diel variation of blood metabolites in 4 volunteers. Samples were taken after overnight fast without breakfast at 9:00; 10:00, 13:00 and before lunch on the first day. Volunteers had lunches and dinners as usual on that day. On the second day after overnight fast, the blood was sampled again at 9:00. During these short periods, the great majority of metabolites hardly fluctuated (117 from 126 metabolites varied less than 2.5-fold on average in four volunteers, FIG. 5A). ATP and ergothioneine hardly varied, although individual ergothioneine levels were distinct. In contrast, four variable compounds fluctuated considerably over 24 hr. Metabolites such as glycochenodeoxycholate, tetradecanoyl-carnitine, 4-aminobenzoate, and caffeine, vary widely, depending upon daily consumption of food, drink, supplements, and medications. Our results are consistent with those previously reported. These daily variable compounds were found in both plasma and RBC (NPL 4).

Determination of Individual CVs for Each Metabolite

We performed metabolomic analyses of blood samples donated by 30 volunteers. Data on compound enrichment in RBCs are consistent with our previous report (NPL 4). The separation of RBCs from WBCs by Ficoll gradient centrifugation confirmed that metabolites and their levels were similar in RBCs and WBCs (NPL 4). Since WBCs make up only a small portion (<1%) of blood volume in healthy individuals, our current results should not be affected by WBC contamination.

Procedures for LC-MS analysis and for obtaining and validating CVs are detailed above. Methods used to determine CVs are briefly described below. First, we tested the effect of sample handling on within-sample variation. To accomplish this, the same blood sample was injected 3 times into the LC-MS. The within-sample CVs (designated as CVwi; FIG. 6A) were less than 0.1 in most cases. These exceptions appear to be labile during LC-MS.

Second, we examined sample-to-sample variation caused by sample preparation. For this purpose, three samples were independently prepared from the same blood sample, and CVs of compounds were determined (CVss) (FIG. 6B). CVss of HEPES and PIPES (internal standards) in blood samples were very small (0.04˜0.08) as they are inert, non-reactive compounds. The great majority of blood compound CVss were less than 0.3 (Table 1). CVss of ten compounds were exceptional, exceeding 0.3. They may either be affected by sample preparation techniques, or may react with other blood metabolites during sample preparation.

CVs for the entire experimental population of 30 persons were determined for each blood compound (CV₃₀) (FIG. 6C and Table 1). CV₃₀ of blood metabolites from all 30 healthy volunteers (Table 2) were arranged into 6 different value ranges with subcategories for compounds enriched in RBCs or present in whole blood (FIG. 1A). Many RBC-enriched compounds such as ATP, glutathione and sugar-phosphate are virtually absent in plasma, but many plasma compounds are also present in RBC (NPL 4).

Twenty-eight compounds having CV₃₀ less than 0.30 comprise the least variable subset of 126 blood metabolites (FIG. 1B). An additional 28 compounds have CV₃₀ values from 0.3 to 0.4, and belong to the second least variable group. Butyrobetaine, a precursor of carnitine, is enriched in RBCs and belongs to this group (FIG. 5B). The remaining 70 compounds show CV₃₀ values from 0.4 to 2.5. We consider these compounds to be variable. Twenty-two compounds having CV₃₀ from 0.4 to 0.5 are moderately variable. Glucose, 1,5-anhydroglucitol, CDP-choline, and glucosamine belong to this group. Creatinine, used for renal tests, belongs to the second group (CV₃₀=0.3-0.4). The 48 compounds with CV₃₀>0.5 are considered highly variable. They are often methylated or acetylated, or modified with bulky groups such as nucleotides or fatty acids.

Previously Unreported CVs for 51 Human Metabolites

The CV₃₀ values of compounds categorized above are, in many cases, well supported by evidence from the literature. CVs of 46 compounds, mostly standard amino acids and their derivatives, analyzed by LC-MS, GC-MS, and NMR, have been previously reported. Of these 46 compounds, 36 had CVs within ±0.3 of our results (CV₃₀). In the literature, we also found CVs for 71 of our 126 compounds. In those reports, 72% of the CVs (51/71) were similar (±0.3) to ours. Overall, our CV₃₀ for 75/126 compounds (60%) (Table 1) are reasonably consistent with the literature. CVs for the remaining 51 compounds are novel, so far as we know. Many of these 51 compounds (underlined in FIG. 1A and also listed in Table 3) are RBC-enriched, as described below.

We classified 126 compounds into 14 categories based on their molecular structures and functions (Table 1). CVs of 17 detectable standard amino acids and all 10 carnitines have been previously reported. Among 12 nucleosides, nucleobases, and their derivatives, only the CV for dimethyl-xanthine was new. In contrast, all 4 nucleotide-sugar derivatives and most (8/9) sugar phosphate derivatives are likewise novel. Their novelty reflects the fact that these compounds are enriched in RBCs. For other categories, some CVs are new: methylated amino acids (8/13), nucleotides (7/11), vitamins and coenzymes (2/5), sugars and derivatives (3/6), organic acids (6/10), acetylated amino acids (5/7), other amino acid derivatives (4/16), choline derivatives (2/3), anti-oxidants (1/3). Many of these are also RBC-enriched. It is curious that methylated amino acids are accumulated in RBCs.

TABLE 3
List of 51 identified metabolites CVs not reported

| | |
|---|---|
| Category/compound | RBC enriched |

Nucleotides (7)

| | |
|---|---|
| CTP | |
| GDP | X |
| GMP | X |
| IMP | X |
| UDP | X |
| UMP | X |
| UTP | |

Nucleosides, nucleobases and derivatives (1)

Dimethyl-xanthine

Vitamins, Coenzymes (2)

| | |
|---|---|
| 4-Aminobenzate | |
| Nicotinamide | X |

Nucleotide-sugar derivatives (4)

| | |
|---|---|
| GDP-glucose | X |
| UDP-acetyl-glucosamine | X |
| UDP-glucose | X |
| UDP-glucuronate | X |

Sugar phosphates (8)

| | |
|---|---|
| 6-Phosphogluconate | X |
| Diphospho-glycerate | X |
| Fructose-6-phosphate | X |
| Glucose-6-phosphate | X |
| Glyceraldehyde-3-phosphate | X |
| Glycerol-2-phosphate | X |
| Pentose-phosphate | X |
| Phosphoglycerate | X |

Sugars and derivatives (3)

| | |
|---|---|
| Gluconate | X |
| Glucosamine | |
| N-Acetyl-D-glucosamine | X |

Organic acids (6)

| | |
|---|---|
| cis-Aconitate | |
| Citramalate | X |
| Glutarate | |
| Glycerate | |
| Malate | X |
| Succinate | X |

Methylated amino acids (8)

| | |
|---|---|
| Butyro-betaine | X |
| Dimethyl-lysine | |
| Methyl-lysine | |
| S-methyl-ergothioneine | X |
| Trimethyl-histidine (hercynine) | X |
| Trimethyl-phenylalanine | X |
| Trimethyl-tryptophan (hypaphorine) | X |
| Trimethyl-tyrosine | X |

Acetylated amino acids (5)

| | |
|---|---|
| N-Acetyl-(iso)leucine | |
| N-Acetyl-aspartate | |
| N-Acetyl-glutamate | X |
| N₂-Acetyl-lysine | |
| N₆-Acetyl-lysine | |

Other amino acid derivatives (4)

| | |
|---|---|
| 4-Guanidinobutanoate | |
| Acetyl-carnosine | |
| Arginino-succinate | |
| Phosphocreatine | X |

Choline derivatives (2)

CDP-choline
CDP-ethanolamine

Antioxidant (1)

| | |
|---|---|
| Ophthalmic acid | X |

Ergothioneine-Related, Glycolytic, and Methylated Compounds are Correlated

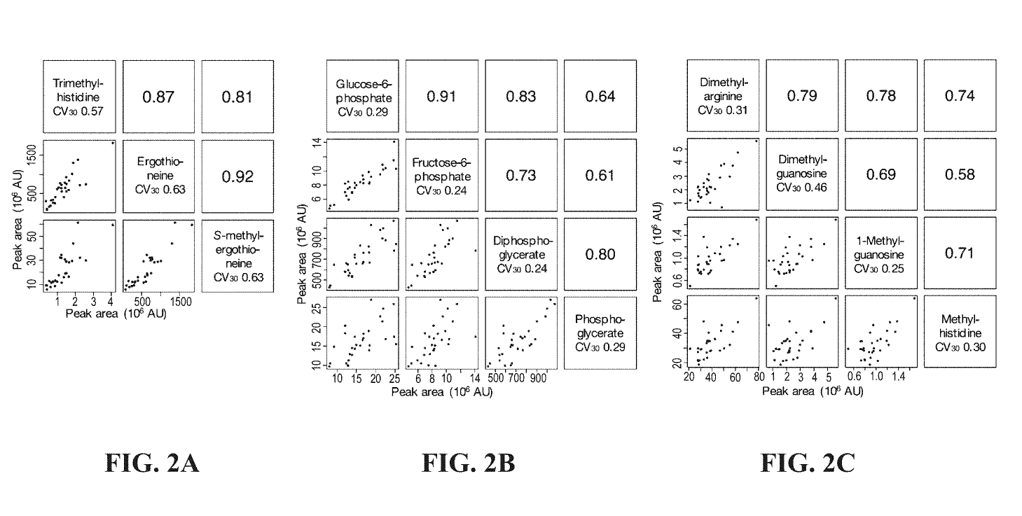

It is interesting that levels of some functionally related blood metabolites are correlated. We first examined whether correlations exist between trimethyl-histidine, ergothioneine, and S-methyl-ergothioneine, as they are structurally related, and the former two compounds are linked in a biochemical pathway. Abundance of these compounds is very strongly, positively correlated (r2=0.81˜0.92, FIG. 2A).

Second, potential correlations among RBC-enriched glucose-6-phosphate (G-6-P), fructose-6-phosphate (F-6-P), diphospho-glycerate (DG) and phosphoglycerate (PG) were examined (FIG. 2B). Very strong correlations were found between G-6-P and F-6-P, between DG and PG, between F-6-P and DG, and between G-6-P and DG. These RBC compounds are components of the glycolytic pathway.

Third, correlations among methylated compounds, dimethyl-arginine (DA), dimethyl-guanosine (DGU), 1-methyl-guanosine (1MG) and methyl-histidine (MH) were also evaluated. DA abundance is strongly and positively correlated with that of DGU, 1MG, and MH (FIG. 2C). In addition, 1MG is also positively correlated to DGU and MH. These results suggest that the levels of some methylated compounds are linked to the same anabolic or catabolic pathways. Consistently, all these compounds are abundant in both RBCs and plasma. Metabolite variations among individuals are thus coordinated in the terms of pathways such as for ergothioneine, glycolysis and methylation.

Metabolites with Low CVs May have Vital Functions

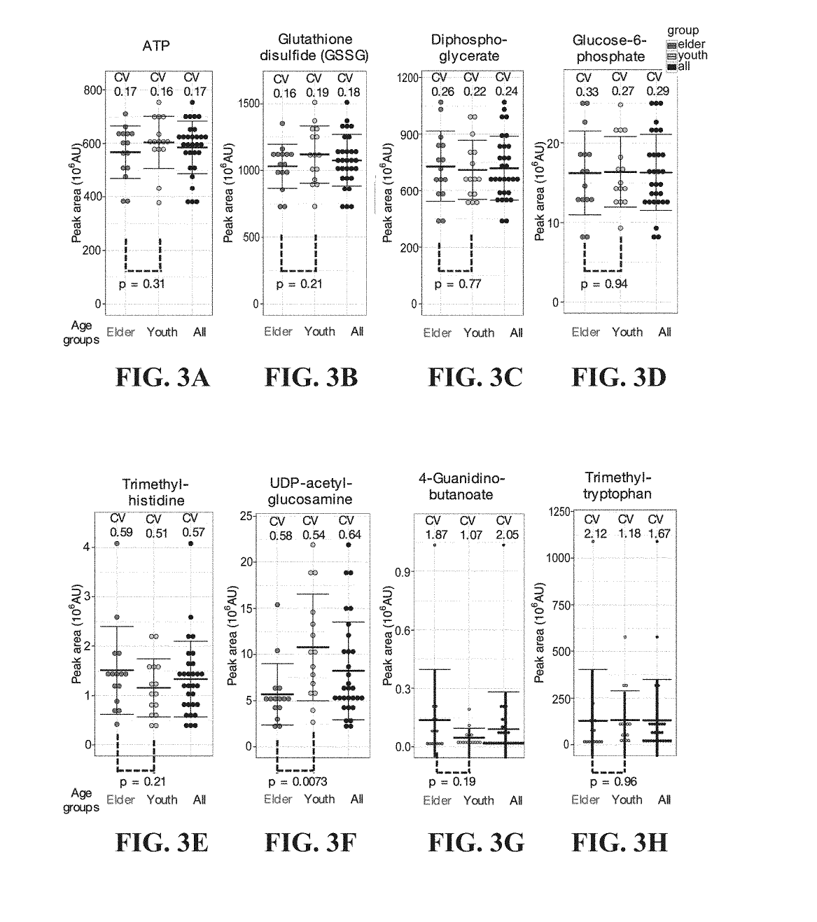

Among the 51 novel CV compounds, 19 showed low CV₃₀<0.4; of these 16 were enriched in RBCs (FIG. 1A, Table 1). They include sugar phosphates, sugar-nucleotide-derivatives, sugars and derivatives, and organic acids involved in ATP production. Compounds with low CVs likely support fundamental RBC functions. CVs of ATP (CV₃₀=0.17) and glutathione disulfide (CV₃₀=0.18) are low and no significant difference was found between elderly and young subjects (FIG. 3A-B). ATP and glutathione are vital as an energy source and an anti-oxidant, respectively, so their concentrations in RBCs may be tightly regulated, with little age-specific variation. A similar situation is seen for two sugar phosphates, diphosphoglycerate (CV₃₀=0.24) and glucose-6-phosphate (CV₃₀=0.29, FIG. 3C-D). It is likely that levels of these key metabolic compounds with small CVs, (ATP, NAD+, standard amino acids, and nucleotides) may be tightly regulated, as they are essential to physiological homeostasis. In other words, small CV compounds might be good candidates for health check indices, provided that measurements are accurate.

Glyceraldehyde-3-phosphate (G-3-P), an essential glycolytic metabolite may be an exception. It has a high CV₃₀ (FIG. 5B). Levels of this compound vary considerably from individual to individual. It is an unstable compound (CVss, 0.49), however, so the high CV₃₀ (0.99) has to be taken cautiously. The enzyme, glyceraldehyde-3-phosphate dehydrogenase, is known to be important in energy metabolism of cancer cells.

Unreported Compounds with High CVs are Often Modified, Implicating Life Style Differences and Dietary Habits

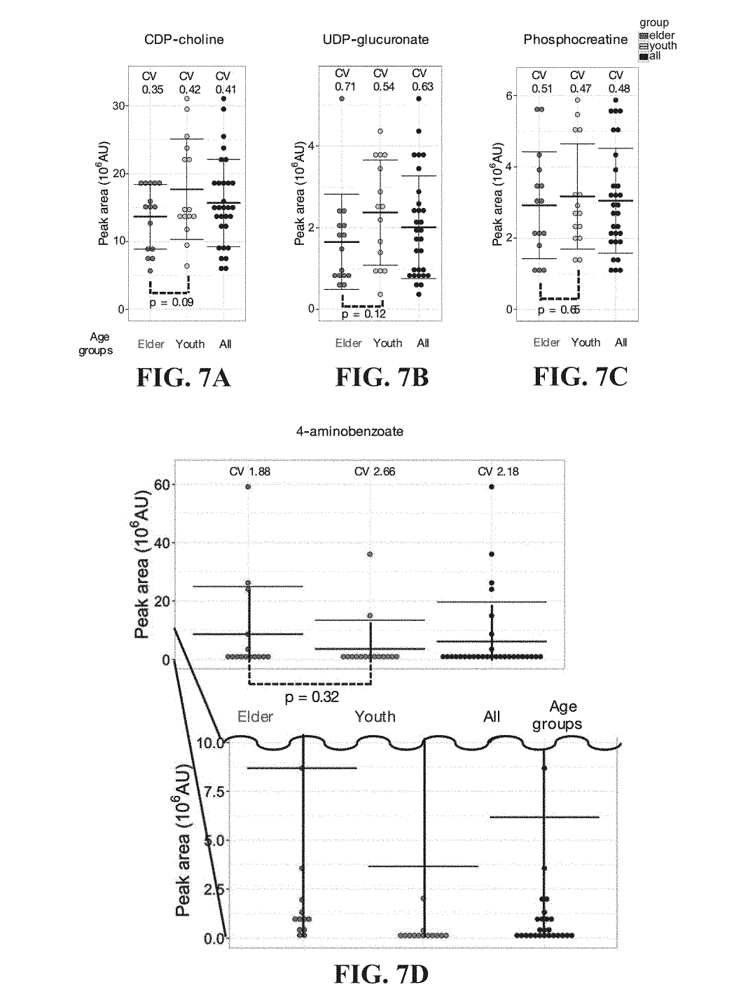

Ten blood metabolites, such as CDP-choline and phosphocreatine, which have not been reported previously, show moderate 0.4˜0.5 CV₃₀ variation (FIG. 1A, FIGS. 7A and 7C). Thirteen compounds show still higher 0.5˜0.7 CV₃₀ (trimethyl-histidine CV₃₀=0.57; FIG. 3E). Nine of them are RBC-enriched, containing nucleotide-sugar and trimethylated derivatives. Their CVs have not been reported previously in blood of healthy individuals. RBC-enriched UDP-glucuronate (CV₃₀=0.64, FIG. 7B) is an intermediate between glucuronides and UDP-glucose. UDP-acetyl-glucosamine (CV₃₀=0.64, FIG. 3F), a substrate for N-acetyl-glucosamine transferase, is a precursor for proteoglycan and glycolipid synthesis. Abundances of UDP-acetyl-glucosamine and UDP-glucuronate show some differences between young and elderly subjects (p-value, 0.0073 and 0.12, respectively; see below).

Compounds showing higher CV₃₀ (0.7-2.5) comprise the most variable group (e.g., 4-guanidino-butanoate CV₃₀ 2.05; trimethyl-tryptophan CV₃₀ 1.67) (FIG. 3G-H). Nine of these have not been reported previously. Four are methylated amino acids, three of which are tri-methylated. Methylated amino acids are enriched in RBCs, whereas acetylated amino acids are found in both plasma and RBCs. The reason for this distinction is unclear. Many of the most variable compounds found are modified amino acids, possibly appropriate as marker compounds related to life style, especially dietary habits.

4-aminobenzoate (also called PABA) data are curious. Its CV₃₀ is very high (2.18). Five people had high levels of PABA, while in all others the level was low or barely detectable (FIG. 7D). PABA is a precursor for vitamin B9 in animals, and in plants and bacteria for folate, but PABA is not essential for humans. This very large variable abundance may reflect dietary or other unknown individual differences.

Age-Related Compounds Revealed by CV Measurements

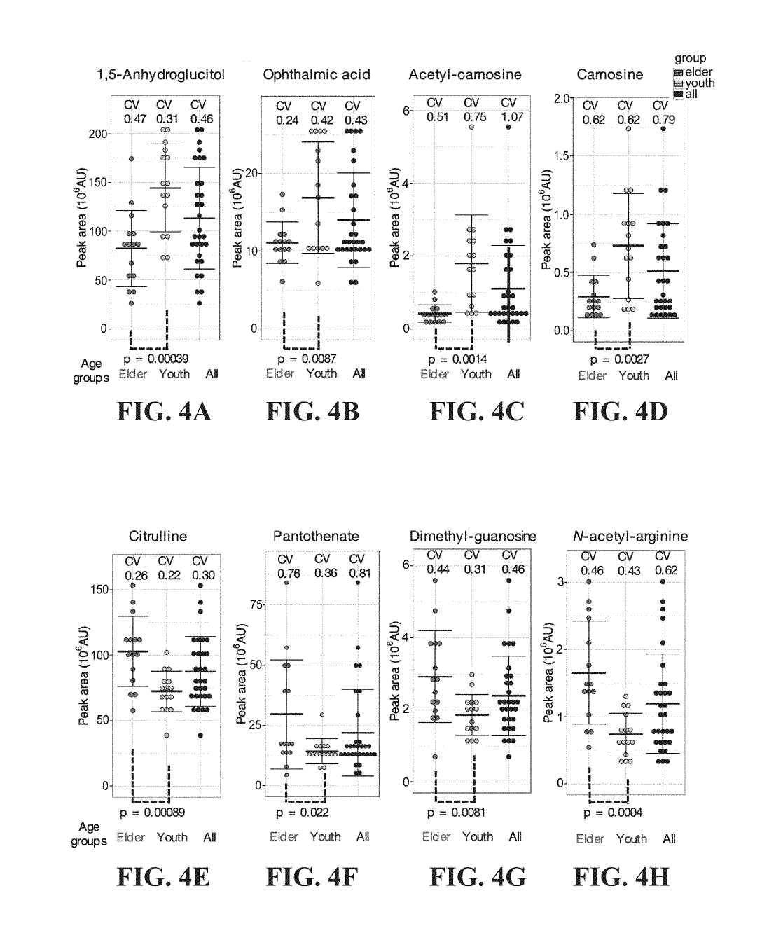

Among 126 compounds analyzed, the great majority showed similar CV levels in young and old people. We found 43 compounds that differed significantly between the two age groups. For example, 1,5-anhydroglucitol (FIG. 4A), known as a glycemic marker (7), shows strikingly lower levels in healthy elderly compared to healthy youths (p=0.00039). Note that none of 30 volunteers are diabetic patient (see the values of HbA1c and glucose in their blood test in Table 2). 1,5-anhydroglucitol, a monosaccharide, is normally re-absorbed back into the blood via the kidneys, but this compound is competitive to glucose for re-absorption so that in diabetic patients containing high glucose in blood, the abundance of 1,5-anhydroglucitol is low. A possible interpretation is that healthy elderly people may gradually lose the ability to re-absorb 1,5-anhydroglucitol, releasing it into urine, with a concomitant decrease in blood.

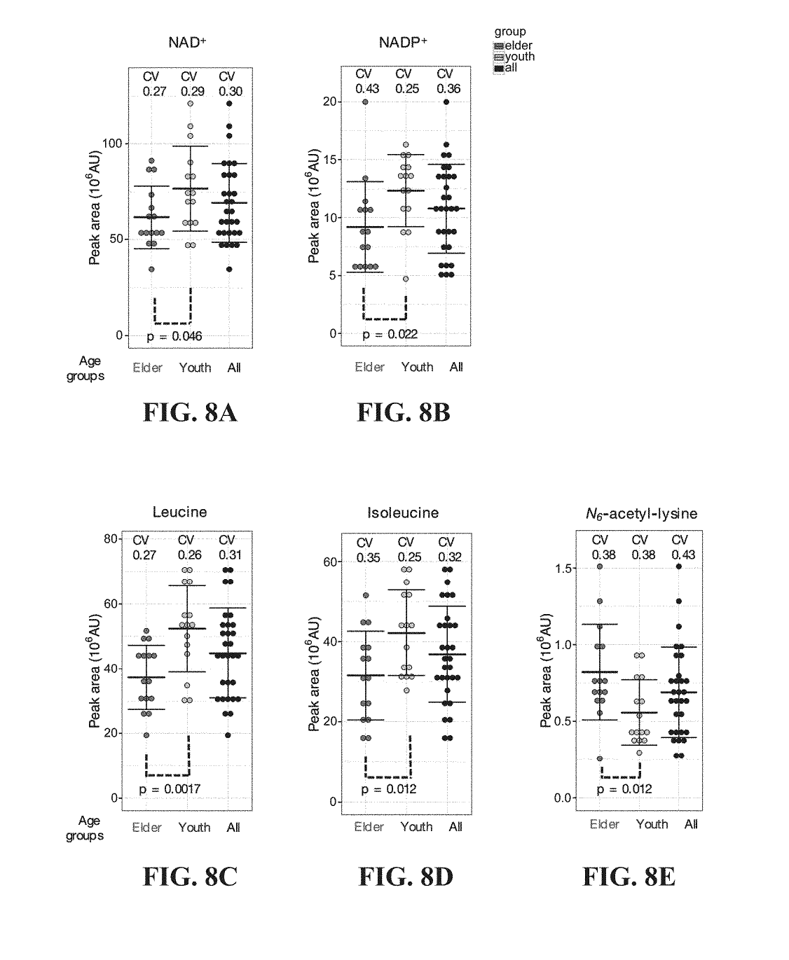

Ophthalmic acid, a tripeptide analog of glutathione, shows impressive difference between young and elderly, (much less in elderly blood; p-value 0.0087; FIG. 4B). Similarly, the levels of two oxidant scavengers, acetyl-carnosine (p=0.0014, FIG. 4C) and carnosine (p=0.0027, FIG. 4D) related dipeptides containing beta-alanine and histidine, are clearly less abundant in elderly. The same holds true for two redox coenzymes enriched in RBCs, NAD+(p=0.046) and NADP+(p=0.022) (FIG. 8A-B), suggesting that the redox metabolism in elderly RBCs might be somewhat declined. Leucine and isoleucine, however, may play a distinct role for supporting skeletal muscle activity in elderly (8) so that their decrease in elderly blood metabolites (p=0.0017 and 0.012, respectively; FIG. 8C-D) might suggest their decrease in blood due to aging. The level of UDP-acetyl-glucosamine that is probably also unrelated to anti-oxidants also decrease in elderly blood (p=0.0073, FIG. 3F). Since this compound is important for growth and proliferation, its decline might also accelerate aging. In short, elderly blood may have reduced anti-oxidants, redox and nutrients required for vigorous body activities.

On the other hand, levels of citrulline (p=0.00089, FIG. 4E), pantothenate (p=0.022, FIG. 4F), dimethyl-guanosine (p=0.0081, FIG. 4G), N-acetyl-arginine (p=0.0004, FIG. 4H) and N6-acetyl-lysine (p=0.012, FIG. 8E) are clearly more abundant in the blood of elderly subjects. Pantothenate is a precursor of CoA, an important coenzyme involved in the TCA cycle and beta-oxidation. Citrulline is the initial metabolite of the urea cycle. Dimethyl-guanosine is a urinary nucleoside, presenting high levels in plasma of uremic patients (6). In patients deficient in arginase (the last enzyme of the urea cycle), N-acetyl-arginine concentrations are >4× higher than normal (4). Hence increased citrulline and N-acetyl-arginine suggest an impaired urea cycle. A possible interpretation of these results is that the excretion of urea cycle metabolites into urine may be somewhat compromised in the elderly. Decreased blood 1,5-anhydroglucitol may also be linked to weakened renal function. Abundant pantothenate in elderly subjects suggests that CoA biosynthesis may be slightly impaired.

We obtained data suggesting that ketoleucine and ketoisoleucine which are degradation metabolites of leucine and isoleucine are significantly decreased in the elderly. It is well known that leucine and isoleucine which are branched amino acids are metabolized in skeletal muscle and brain. Especially ATP is converted to IMP through ADP and AMP during exercise in muscle. Toxic ammonia is produced in this process. Ammonia and glutamic acid combine with glutamine synthetase and are converted into nontoxic glutamine. During exercise, the branched amino acids react with 2-ketoglutamate in the presence of aminotransferase to produce glutamic acid which is necessary for treatment of ammonia. Ketoleucine and ketoisoleucine are produced by this enzymatic reaction. Finally, it is converted to acetyl CoA or succinyl CoA and used for citric acid cycle. Therefore, it is reasonable that ketoleucine and ketoisoleucine are lower in elder which reflects a decrease in muscle mass and momentum. Ketoleucine and ketoisoleucine can be used as an aging marker.

In addition to the blood metabolites described above, 27 compounds were found that showed a significant difference between the elderly and young people. Blood metabolites that differed between the elderly and young people are summarized in the tables below.

TABLE 4

| | | Blood | | | | | |
|---|---|---|---|---|---|---|---|
| | | | Average of peak area | | Fold-change | | Up/Down |
| No. | Name | p-value | Young | Old | Young/old | Old/young | in old |
| 1 | 1,5-Anhydroglucitol | 0.00039 | 1.4E+08 | 8.2E+07 | 1.75 | 0.57 | ↓ |
| 2 | Acetyl-carnosine | 0.00141 | 1.8E+06 | 4.1E+05 | 4.33 | 0.23 | ↓ |
| 3 | Adenosine | 0.05208 | 9.0E+05 | 6.4E+05 | 1.41 | 0.71 | ↓ |
| 4 | Aspartate | 0.62048 | 1.9E+07 | 2.1E+07 | 0.91 | 1.10 | ↑ |
| 5 | Carnosine | 0.00269 | 7.3E+05 | 2.9E+05 | 2.48 | 0.40 | ↓ |
| 6 | CDP-choline | 0.08815 | 1.8E+07 | 1.4E+07 | 1.29 | 0.77 | ↓ |
| 7 | CDP-ethanolamine | 0.07673 | 1.2E+06 | 8.8E+05 | 1.33 | 0.75 | ↓ |
| 8 | Citrulline | 0.00089 | 7.2E+07 | 1.0E+08 | 0.70 | 1.42 | ↑ |
| 9 | Creatine | 0.57527 | 1.4E+08 | 1.3E+08 | 1.06 | 0.94 | ↓ |
| 10 | CTP | 0.07804 | 3.1E+05 | 2.5E+05 | 1.23 | 0.81 | ↓ |
| 11 | Dimethyl-arginine | 0.07902 | 3.5E+07 | 4.3E+07 | 0.82 | 1.23 | ↑ |
| 12 | Dimethyl-guanosine | 0.00806 | 1.9E+06 | 2.9E+06 | 0.64 | 1.57 | ↑ |
| 13 | Diphospho-glycerate | 0.76552 | 7.1E+08 | 7.3E+08 | 0.97 | 1.03 | ↑ |
| 14 | Fructose-6-phosphate | 0.61486 | 8.6E+06 | 8.2E+06 | 1.05 | 0.96 | ↑ |
| 15 | Glucose-6-phosphate | 0.94010 | 1.6E+07 | 1.6E+07 | 1.01 | 0.99 | ↑ |
| 16 | Glutamate | 0.29000 | 5.7E+07 | 6.4E+07 | 0.90 | 1.12 | ↑ |
| 17 | Glutarate | 0.65314 | 1.0E+07 | 9.5E+06 | 1.05 | 0.95 | ↓ |
| 18 | Glutathione disulfide (GSSG) | 0.21086 | 1.1E+09 | 1.0E+09 | 1.09 | 0.92 | ↓ |
| 19 | Glycerol-2-phosphate | 0.32112 | 3.2E+06 | 3.6E+06 | 0.89 | 1.12 | ↑ |
| 20 | Glycerophosphocholine | 0.65640 | 7.3E+07 | 6.8E+07 | 1.08 | 0.92 | ↓ |
| 21 | Histidine | 0.06035 | 4.2E+07 | 3.7E+07 | 1.14 | 0.88 | ↓ |
| 22 | Isoleucine | 0.01239 | 4.2E+07 | 3.2E+07 | 1.34 | 0.75 | ↓ |
| 23 | Leucine | 0.00172 | 5.2E+07 | 3.7E+07 | 1.40 | 0.71 | ↓ |
| 24 | N-Acetyl-(iso)leucine | 0.05635 | 6.3E+05 | 9.9E+05 | 0.64 | 1.56 | ↑ |
| 25 | N-Acetyl-arginine | 0.00040 | 7.4E+05 | 1.7E+06 | 0.44 | 2.25 | ↑ |
| 26 | N2-Acetyl-lysine | 0.03304 | 5.8E+05 | 8.6E+05 | 0.67 | 1.49 | ↑ |
| 27 | N6-Acetyl-lysine | 0.01206 | 5.6E+05 | 8.2E+05 | 0.68 | 1.47 | ↑ |
| 28 | NAD+ | 0.04573 | 7.7E+07 | 6.2E+07 | 1.24 | 0.81 | ↓ |
| 29 | NADP+ | 0.02225 | 1.2E+07 | 9.2E+06 | 1.34 | 0.74 | ↓ |
| 30 | Ophthalmic acid | 0.00870 | 1.7E+07 | 1.1E+07 | 1.52 | 0.66 | ↓ |
| 31 | Pantothenate | 0.02163 | 1.4E+07 | 3.0E+07 | 0.48 | 2.07 | ↑ |
| 32 | Pentose-phosphate | 0.04944 | 1.6E+06 | 1.2E+06 | 1.28 | 0.78 | ↓ |
| 33 | Phenylalanine | 0.04111 | 2.8E+08 | 3.2E+08 | 0.88 | 1.14 | ↑ |
| 34 | Phosphocreatine | 0.65072 | 3.2E+06 | 2.9E+06 | 1.09 | 0.92 | ↓ |
| 35 | S-Adenosyl-homocysteine | 0.04362 | 2.5E+06 | 1.3E+06 | 1.89 | 0.53 | ↓ |
| 36 | Serine | 0.20154 | 1.1E+06 | 9.2E+05 | 1.17 | 0.85 | ↓ |
| 37 | Tryptophan | 0.21639 | 1.1E+08 | 1.0E+08 | 1.11 | 0.90 | ↓ |
| 38 | Tyrosine | 0.05071 | 7.4E+07 | 8.9E+07 | 0.83 | 1.21 | ↑ |
| 39 | UDP-acetyl-glucosamine | 0.00732 | 1.1E+07 | 5.7E+06 | 1.89 | 0.53 | ↓ |
| 40 | UDP-glucose | 0.08809 | 3.3E+07 | 2.8E+07 | 1.16 | 0.86 | ↓ |
| 41 | UTP | 0.46353 | 7.0E+05 | 6.2E+05 | 1.13 | 0.89 | ↓ |
| 42 | Keto(iso)leucine | 0.00060 | 7.1E+07 | 4.8E+07 | 1.48 | 0.67 | ↓ |
| 43 | Ketovaline | 0.42084 | 1.9E+06 | 1.7E+06 | 1.13 | 0.89 | ↓ |

TABLE 5

| | | Plasma | | | | | |
|---|---|---|---|---|---|---|---|
| | | | Average of peak area | | Fold-change | | Up/Down |
| No. | Name | p-value | Young | Old | Young/old | Old/young | in old |
| 1 | 1,5-Anhydroglucitol | 0.00284 | 1.5E+08 | 8.9E+07 | 1.67 | 0.60 | ↓ |
| 2 | Acetyl-carnosine | 0.00050 | 4.5E+06 | 1.3E+06 | 3.49 | 0.29 | ↓ |
| 3 | Adenosine | 0.32568 | 2.0E+07 | 2.4E+07 | 0.82 | 1.21 | ↑ |
| 4 | Aspartate | 0.25237 | 1.5E+06 | 1.2E+06 | 1.32 | 0.76 | ↓ |
| 5 | Carnosine | 0.00720 | 8.5E+04 | 3.4E+04 | 2.47 | 0.40 | ↓ |
| 6 | CDP-choline | 0.00584 | 2.0E+07 | 1.0E+07 | 1.97 | 0.51 | ↓ |
| 7 | CDP-ethanolamine | 0.02575 | 1.1E+06 | 6.8E+05 | 1.59 | 0.63 | ↓ |
| 8 | Citrulline | 0.01965 | 8.0E+07 | 1.0E+08 | 0.78 | 1.28 | ↑ |
| 9 | Creatine | 0.01452 | 3.9E+07 | 6.1E+07 | 0.64 | 1.55 | ↑ |
| 10 | CTP | 0.11893 | 3.2E+05 | 2.3E+05 | 1.37 | 0.73 | ↓ |
| 11 | Dimethyl-arginine | 0.13982 | 4.3E+07 | 5.2E+07 | 0.84 | 1.20 | ↑ |
| 12 | Dimethyl-guanosine | 0.00752 | 2.1E+06 | 3.0E+06 | 0.70 | 1.44 | ↑ |
| 13 | Diphospho-glycerate | 0.23739 | 3.5E+05 | 8.1E+05 | 0.43 | 2.33 | ↑ |
| 14 | Fructose-6-phosphate | 0.01239 | 5.4E+05 | 3.8E+05 | 1.43 | 0.70 | ↓ |
| 15 | Glucose-6-phosphate | 0.43891 | 1.1E+06 | 1.0E+06 | 1.11 | 0.90 | ↓ |
| 16 | Glutamate | 0.76910 | 1.9E+07 | 2.0E+07 | 0.96 | 1.04 | ↑ |
| 17 | Glutarate | 0.15218 | 9.4E+06 | 1.1E+07 | 0.85 | 1.17 | ↑ |
| 18 | Glutathione disulfide (GSSG) | 0.00001 | 2.3E+06 | 1.1E+06 | 2.14 | 0.47 | ↓ |
| 19 | Glycerol-2-phosphate | 0.25221 | 9.2E+05 | 7.9E+05 | 1.16 | 0.86 | ↓ |
| 20 | Glycerophosphocholine | 0.00288 | 4.7E+07 | 3.4E+07 | 1.38 | 0.72 | ↓ |
| 21 | Histidine | 0.00841 | 6.6E+07 | 5.4E+07 | 1.22 | 0.82 | ↓ |
| 22 | Isoleucine | 0.25159 | 4.1E+07 | 3.5E+07 | 1.16 | 0.86 | ↓ |
| 23 | Leucine | 0.03604 | 5.1E+07 | 4.0E+07 | 1.27 | 0.79 | ↓ |
| 24 | N-Acetyl-(iso)leucine | 0.06150 | 5.3E+05 | 8.1E+05 | 0.65 | 1.53 | ↑ |
| 25 | N-Acetyl-arginine | 0.00029 | 1.5E+06 | 3.2E+06 | 0.47 | 2.12 | ↑ |
| 26 | N2-Acetyl-lysine | 0.00100 | 5.3E+05 | 1.1E+06 | 0.49 | 2.04 | ↑ |
| 27 | N6-Acetyl-lysine | 0.04835 | 7.8E+05 | 1.0E+06 | 0.77 | 1.30 | ↑ |
| 28 | NAD+ | 0.01257 | 1.9E+06 | 1.3E+06 | 1.49 | 0.67 | ↓ |
| 29 | NADP+ | ND in plasma | | | | | |
| 30 | Ophthalmic acid | 0.15622 | 1.1E+05 | 4.2E+04 | 2.57 | 0.39 | ↓ |
| 31 | Pantothenate | 0.04830 | 8.9E+06 | 1.7E+07 | 0.51 | 1.97 | ↑ |
| 32 | Pentose-phosphate | ND in plasma | | | | | |
| 33 | Phenylalanine | 0.15976 | 3.2E+08 | 3.5E+08 | 0.92 | 1.09 | ↑ |
| 34 | Phosphocreatine | 0.00868 | 1.8E+06 | 1.1E+06 | 1.58 | 0.63 | ↓ |
| 35 | S-Adenosyl-homocysteine | ND in plasma | | | | | |
| 36 | Serine | 0.01197 | 1.3E+06 | 9.4E+05 | 1.40 | 0.71 | ↓ |
| 37 | Tryptophan | 0.02158 | 1.8E+08 | 1.5E+08 | 1.24 | 0.80 | ↓ |
| 38 | Tyrosine | 0.02555 | 7.2E+07 | 8.8E+07 | 0.82 | 1.21 | ↑ |
| 39 | UDP-acetyl-glucosamine | 0.00491 | 1.8E+06 | 9.2E+05 | 2.01 | 0.50 | ↓ |
| 40 | UDP-glucose | 0.01380 | 1.1E+06 | 6.3E+05 | 1.75 | 0.57 | ↓ |
| 41 | UTP | 0.00021 | 8.9E+05 | 4.3E+05 | 2.05 | 0.49 | ↓ |
| 42 | Keto(iso)leucine | 0.00074 | 9.7E+07 | 6.1E+07 | 1.60 | 0.62 | ↓ |
| 43 | Ketovaline | 0.61388 | 2.3E+06 | 2.1E+06 | 1.12 | 0.90 | ↓ |

TABLE 6

| | | RBC | | | | | |
|---|---|---|---|---|---|---|---|
| | | | Average of peak area | | Fold-change | | Up/Down |
| No. | Name | p-value | Young | Old | Young/old | Old/young | in old |
| 1 | 1,5-Anhydroglucitol | 0.01967 | 1.2E+08 | 8.2E+07 | 1.47 | 0.68 | ↓ |
| 2 | Acetyl-carnosine | 0.00103 | 7.3E+05 | 1.3E+05 | 5.63 | 0.18 | ↓ |
| 3 | Adenosine | 0.02210 | 7.1E+05 | 4.5E+05 | 1.59 | 0.63 | ↓ |
| 4 | Aspartate | 0.03043 | 2.3E+07 | 3.8E+07 | 0.61 | 1.63 | ↑ |
| 5 | Carnosine | 0.03293 | 5.3E+05 | 3.3E+05 | 1.60 | 0.63 | ↓ |
| 6 | CDP-choline | 0.28626 | 1.2E+07 | 1.1E+07 | 1.16 | 0.86 | ↓ |
| 7 | CDP-ethanolamine | 0.06612 | 1.1E+06 | 7.9E+05 | 1.35 | 0.74 | ↓ |
| 8 | Citrulline | 0.00358 | 6.4E+07 | 9.1E+07 | 0.71 | 1.41 | ↑ |
| 9 | Creatine | 0.53974 | 1.6E+08 | 1.7E+08 | 0.93 | 1.07 | ↑ |
| 10 | CTP | 0.00502 | 2.1E+05 | 1.3E+05 | 1.57 | 0.64 | ↓ |
| 11 | Dimethyl-arginine | 0.02386 | 2.9E+07 | 3.8E+07 | 0.75 | 1.33 | ↑ |
| 12 | Dimethyl-guanosine | 0.00808 | 1.8E+06 | 2.9E+06 | 0.60 | 1.66 | ↑ |
| 13 | Diphospho-glycerate | 0.01302 | 9.4E+08 | 1.2E+09 | 0.80 | 1.26 | ↑ |
| 14 | Fructose-6-phosphate | 0.11491 | 9.6E+06 | 1.1E+07 | 0.85 | 1.18 | ↑ |
| 15 | Glucose-6-phosphate | 0.01845 | 2.0E+07 | 2.7E+07 | 0.76 | 1.32 | ↑ |
| 16 | Glutamate | 0.04504 | 7.1E+07 | 9.1E+07 | 0.78 | 1.28 | ↑ |
| 17 | Glutarate | 0.04222 | 6.1E+06 | 8.3E+06 | 0.73 | 1.37 | ↑ |
| 18 | Glutathione disulfide (GSSG) | 0.75591 | 1.5E+09 | 1.5E+09 | 1.01 | 0.99 | ↓ |
| 19 | Glycerol-2-phosphate | 0.00495 | 3.3E+06 | 5.3E+06 | 0.63 | 1.58 | ↑ |
| 20 | Glycerophosphocholine | 0.63225 | 7.6E+07 | 6.9E+07 | 1.10 | 0.91 | ↓ |
| 21 | Histidine | 0.54058 | 4.2E+07 | 4.0E+07 | 1.05 | 0.95 | ↓ |
| 22 | Isoleucine | 0.52965 | 3.5E+07 | 3.2E+07 | 1.09 | 0.91 | ↓ |
| 23 | Leucine | 0.16558 | 4.9E+07 | 4.0E+07 | 1.22 | 0.82 | ↓ |
| 24 | N-Acetyl-(iso)leucine | 0.01376 | 4.8E+05 | 7.8E+05 | 0.61 | 1.65 | ↑ |
| 25 | N-Acety-arginine | 0.00467 | 4.4E+05 | 9.5E+05 | 0.47 | 2.14 | ↑ |
| 26 | N2-Acetyl-lysine | 0.09706 | 7.4E+05 | 1.0E+06 | 0.74 | 1.35 | ↑ |
| 27 | N6-Acetyl-lysine | 0.06019 | 4.6E+05 | 6.2E+05 | 0.73 | 1.37 | ↑ |
| 28 | NAD+ | 0.23505 | 1.2E+08 | 1.1E+08 | 1.11 | 0.90 | ↓ |
| 29 | NADP+ | 0.08244 | 2.1E+07 | 1.7E+07 | 1.24 | 0.81 | ↓ |
| 30 | Ophthalmic acid | 0.15259 | 3.2E+07 | 2.6E+07 | 1.20 | 0.83 | ↓ |
| 31 | Pantothenate | 0.02951 | 1.8E+07 | 3.7E+07 | 0.48 | 2.07 | ↑ |
| 32 | Pentose-phosphate | 0.85234 | 2.1E+06 | 2.2E+06 | 0.98 | 1.02 | ↑ |
| 33 | Phenylalanine | 0.04886 | 2.7E+08 | 3.0E+08 | 0.90 | 1.11 | ↑ |
| 34 | Phosphocreatine | 0.91635 | 3.5E+06 | 3.6E+06 | 0.98 | 1.02 | ↑ |
| 35 | S-Adenosyl-homocysteine | 0.07274 | 5.3E+06 | 3.2E+06 | 1.66 | 0.60 | ↓ |
| 36 | Serine | 0.67158 | 1.3E+06 | 1.2E+06 | 1.10 | 0.91 | ↓ |
| 37 | Tryptophan | 0.37775 | 8.4E+07 | 7.9E+07 | 1.07 | 0.93 | ↓ |
| 38 | Tyrosine | 0.00327 | 7.1E+07 | 9.2E+07 | 0.77 | 1.30 | ↑ |
| 39 | UDP-acetyl-glucosamine | 0.10343 | 1.2E+07 | 8.2E+06 | 1.45 | 0.69 | ↓ |
| 40 | UDP-glucose | 0.20271 | 4.1E+07 | 4.9E+07 | 0.85 | 1.17 | ↑ |
| 41 | UTP | 0.33022 | 5.9E+05 | 4.9E+05 | 1.22 | 0.82 | ↓ |
| 42 | Keto(iso)leucine | 0.00064 | 4.7E+07 | 3.2E+07 | 1.47 | 0.68 | ↓ |
| 43 | Ketovaline | 0.01104 | 1.9E+06 | 1.4E+06 | 1.40 | 0.72 | ↓ |

TABLE 7

| | | Up/Down | RBC | |
|---|---|---|---|---|
| No. | Name | in old | enriched | RBC/plasma |
| 1 | 1,5-Anhydroglucitol | B↓ P↓ R↓ | | 0.9 ± 0.3 |
| 2 | Acetyl-carnosine | B↓ P↓ R↓ | | 0.1 ± 0.0 |
| 3 | Adenosine | R↓ | | 0.0 ± 0.0 |
| 4 | Aspartate | R↑ | ◯ | 41.1 ± 49.4 |
| 5 | Carnosine | B↓ P↓ R↓ | ◯ | 10.8 ± 8.5 |
| 6 | CDP-choline | P↓ | | 1.2 ± 1.4 |
| 7 | CDP-ethanolamine | P↓ | | 1.5 ± 1.8 |
| 8 | Citrulline | B↑ P↑ R↑ | | 0.9 ± 0.2 |
| 9 | Creatine | P↑ | ◯ | 3.9 ± 2.0 |
| 10 | CTP | R↓ | | 0.9 ± 1.2 |
| 11 | Dimethyl-arginine | R↑ | | 0.7 ± 0.2 |
| 12 | Dimethyl-guanosine | B↑ P↑ R↑ | | 0.9 ± 0.3 |
| 13 | Diphospho-glycerate | R↑ | ◯ | 4695 ± 2888 |
| 14 | Fructose-6-phosphate | P↓ | ◯ | 28.1 ± 18.9 |
| 15 | Glucose-6-phosphate | R↑ | ◯ | 26.1 ± 19.1 |
| 16 | Glutamate | R↑ | ◯ | 4.9 ± 2.9 |
| 17 | Glutarate | R↑ | | 0.8 ± 0.5 |
| 18 | Glutathione disulfide (GSSG) | P↓ | ◯ | 1604 ± 2377 |
| 19 | Glycerol-2-phosphate | R↑ | ◯ | 6.1 ± 4.8 |
| 20 | Glycerophosphocholine | P↓ | | 1.9 ± 1.0 |
| 21 | Histidine | P↓ | | 0.7 ± 0.2 |
| 22 | Isoleucine | B↓ | | 0.9 ± 0.4 |
| 23 | Leucine | B↓ P↓ | | 1.0 ± 0.3 |
| 24 | N-Acetyl-(iso)leucine | R↑ | | 1.1 ± 0.5 |
| 25 | N-Acetyl-arginine | B↑ P↑ R↑ | | 0.3 ± 0.1 |
| 26 | N2-Acetyl-lysine | B↑ P↑ | | 1.3 ± 0.7 |
| 27 | N6-Acetyl-lysine | B↑ P↑ | | 0.7 ± 0.3 |
| 28 | NAD+ | B↓ P↓ | ◯ | 101.4 ± 115.7 |
| 29 | NADP+ | B↓ | ◯ | ND in plasma |
| 30 | Ophthalmic acid | B↓ | ◯ | 914.9 ± 991.8 |
| 31 | Pantothenate | B↑ P↑ R↑ | ◯ | 2.1 ± 1.0 |
| 32 | Pentose-phosphate | B↓ | ◯ | ND in plasma |
| 33 | Phenylalanine | B↑ R↑ | | 0.9 ± 0.1 |
| 34 | Phosphocreatine | P↓ | ◯ | 2.8 ± 1.9 |
| 35 | S-Adenosyl-homocysteine | B↓ | ◯ | ND in plasma |
| 36 | Serine | P↓ | | 1.2 ± 0.7 |
| 37 | Tryptophan | P↓ | | 0.5 ± 0.1 |
| 38 | Tyrosine | P↑ R↑ | | 1.0 ± 0.2 |
| 39 | UDP-acetyl-glucosamine | B↓ P↓ | ◯ | 12.8 ± 15.2 |
| 40 | UDP-glucose | P↓ | ◯ | 227.7 ± 694.4 |
| 41 | UTP | P↓ | | 1.6 ± 3.1 |
| 42 | Keto(iso)leucine | B↓ P↓ R↓ | | 0.6 ± 0.1 |
| 43 | Ketovaline | R↓ | | 0.8 ± 0.1 |

Correlations Among Age-Related Compounds

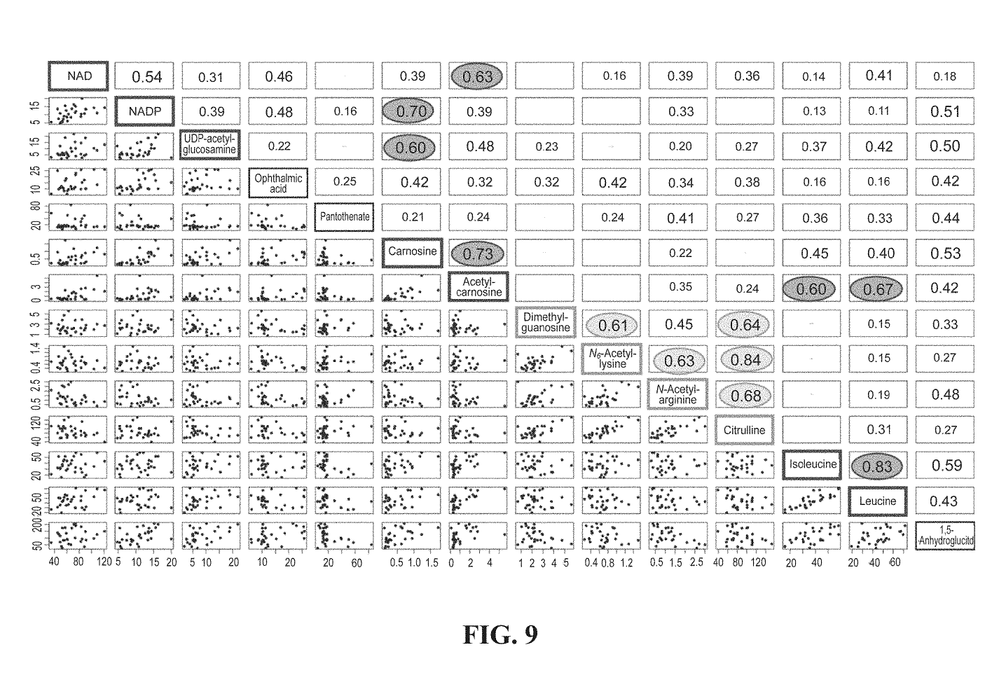

At first, we found 12 pairs of 14 age-related compounds (1,5-Anhydroglucitol, N-Acetyl-arginine, Citrulline, Acetyl-carnosine, Leucine, Ophthalmic acid, N6-Acetyl-lysine, Carnosine, UDP-acetyl-glucosamine, NAD+, NADP+, Isoleucine, Pantothenate and Dimethyl-guanosine) that showed relatively strong correlation coefficients (Pearson's r) (0.60-0.84; Table 8, FIG. 9). Interestingly, such combinations occurred within groups of compounds that either increased or decreased among the elderly. Citrulline content is strongly correlated with N-acetyl-lysine (0.84), and less so with N-acetyl-arginine (0.68) and dimethyl guanosine (0.64) (Table 8). Correlations also exist between N-acetyl-arginine and N-acetyl-lysine (0.63) and between N-acetyl-arginine and dimethyl-guanosine (0.61). These four compounds show increased blood levels in the elderly. We then found correlations (0.6-0.83) among seven compounds that decreased in elderly. Correlations between leucine and isoleucine (0.83) and between carnosine and acetyl-carnosine (0.73) are strong, suggesting that these compounds are correlated because of their close functional relationships. Other closely correlated combinations include carnosine and NADP+, leucine and acetyl carnosine (Table 8; FIG. 9). These results are consistent with a notion that abundances of two distinct groups of age-related compounds (decrease or increase in elderly) are internally correlated, but no correlation exists between the groups. For example, elderly volunteers who have abundant leucine would have also high isoleucine in a high probability, while those elderly have abundant citrulline would have high N6-acetyl-lysine also in a high probability. However, there is no correlation for leucine and citrulline abundances among individuals.

TABLE 8
The pairs of age-related compounds that
show relatively high correlation values.

| Age-related | Age-related | Correlation |
|---|---|---|
| Citrulline | N-acetyl-arginine | 0.68 |
| Citrulline | N-acetyl-lysine | 0.84 |
| Citrulline | Dimethyl-guanosine | 0.64 |
| N-acetyl-arginine | N-acetyl-lysine | 0.63 |
| N-acetyl-lysine | Dimethyl-guanosine | 0.61 |
| Leucine | Isoleucine | 0.83 |
| Leucine | Acetyl-carnosine | 0.67 |
| Isoleucine | Acetyl-carnosine | 0.60 |
| Carnosine | Acetyl-carnosine | 0.73 |
| Acetyl-carnosine | NAD+ | 0.63 |
| Carnosine | UDP-acetylglucosamine | 0.70 |
| Carnosine | NADP+ | 0.70 |

First five pairs of compounds show higher levels in elderly, while the other seven pairs of compounds are more abundant in young persons. See supplementary FIG. 9

We investigated the correlation between 43 age-related compounds. We found 27 pairs of 43 age-related compounds in whole blood that showed relatively strong correlation coefficients (Pearson's r) (0.60-0.91; Table 9). In addition, we found 45 pairs of 43 age-related compounds in plasma that showed relatively strong correlation coefficients (Pearson's r) (0.61-0.85; Table 10). We also found 35 pairs of 43 age-related compounds in RBC that showed relatively strong correlation coefficients (Pearson's r) (−0.64-0.94; Table 11).

TABLE 9
The pairs of age-related compounds in whole blood
that show relatively high correlation values.

| Age-related | Age-related | Correlation |
|---|---|---|
| 1,5-Anhydroglucitol | Keto(iso)leucine | 0.67 |
| Acetylcarnosine | Carnosine | 0.73 |
| Acetylcarnosine | NAD+ | 0.63 |
| Carnosine | NADP+ | 0.70 |
| CDP-choline | CDP-ethanolamine | 0.87 |
| Citrulline | Dimethyl-arginine | 0.67 |
| Citrulline | Dimethyl-guanosine | 0.64 |
| Citrulline | N-Acetyl-arginine | 0.68 |
| Citrulline | N6-Acetyl-lysine | 0.84 |
| Dimethyl-arginine | Dimethyl-guanosine | 0.79 |
| Dimethyl-arginine | N-Acetyl-arginine | 0.61 |
| Dimethyl-arginine | N6-Acetyl-lysine | 0.76 |
| Dimethyl-guanosine | N6-Acetyl-lysine | 0.61 |
| Diphospho-glycerate | Fructose-6-phosphate | 0.73 |
| Diphospho-glycerate | Glucose-6-phosphate | 0.83 |
| Diphospho-glycerate | Glutamate | 0.65 |
| Diphospho-glycerate | Glycerol-phosphate | 0.61 |
| Fructose-6-phosphate | Glucose-6-phosphate | 0.91 |
| Fructose-6-phosphate | Glycerol-phosphate | 0.65 |
| Glucose-6-phosphate | Glycerol-phosphate | 0.65 |
| Glutamate | Glycerol-phosphate | 0.60 |
| Isoleucine | Leucine | 0.83 |
| N-Acetyl-(iso)leucine | N6-Acetyl-lysine | 0.63 |
| N-Acetyl-arginine | N2-Acetyl-lysine | 0.77 |
| N-Acetyl-arginine | N6-Acetyl-lysine | 0.63 |
| N6-Acetyl-lysine | Tyrosine | 0.74 |
| Phenylalanine | Tyrosine | 0.74 |

TABLE 10
The pairs of age-related compounds in plasma
that show relatively high correlation values.

| Age-related | Age-related | Correlation |
|---|---|---|
| 1,5-Anhydroglucitol | Carnosine | 0.61 |
| 1,5-Anhydroglucitol | Keto(iso)leucine | 0.67 |
| Acetylcarnosine | Carnosine | 0.63 |
| Acetylcarnosine | Leucine | 0.62 |
| Acetylcarnosine | Keto(iso)leucine | 0.64 |
| Carnosine | Phosphocreatine | 0.63 |
| Carnosine | UDP-acetyl-glucosamine | 0.66 |
| CDP-choline | CDP-ethanolamine | 0.76 |
| CDP-choline | CTP | 0.67 |
| CDP-choline | Glycerophosphocholine | 0.70 |
| CDP-choline | NAD+ | 0.74 |
| CDP-ethanolamine | CTP | 0.82 |
| CDP-ethanolamine | Fructose-6-phosphate | 0.69 |
| CDP-ethanolamine | Glutathione disulfide | 0.62 |
| | (GSSG) | |
| CDP-ethanolamine | Glycerophosphocholine | 0.79 |
| CDP-ethanolamine | NAD+ | 0.74 |
| CDP-ethanolamine | UTP | 0.70 |
| Citrulline | N6-Acetyl-lysine | 0.63 |
| CTP | Fructose-6-phosphate | 0.71 |
| CTP | Glycerophosphocholine | 0.70 |
| CTP | NAD+ | 0.66 |
| CTP | UTP | 0.76 |
| Dimethyl-arginine | Dimethyl-guanosine | 0.81 |
| Dimethyl-arginine | N-Acetyl-arginine | 0.64 |
| Dimethyl-arginine | N6-Acetyl-lysine | 0.84 |
| Dimethyl-guanosine | N6-Acetyl-lysine | 0.77 |
| Fructose-6-phosphate | Glucose-6-phosphate | 0.64 |
| Fructose-6-phosphate | Glycerophosphocholine | 0.63 |
| Fructose-6-phosphate | UDP-acetyl-glucosamine | 0.68 |
| Fructose-6-phosphate | UDP-glucose | 0.60 |
| Fructose-6-phosphate | UTP | 0.85 |
| Glucose-6-phosphate | Glycerol-phosphate | 0.68 |
| Glucose-6-phosphate | UTP | 0.67 |
| Glutathione disulfide | NAD+ | 0.65 |
| (GSSG) | | |
| Glycerophosphocholine | NAD+ | 0.74 |
| Glycerophosphocholine | UTP | 0.76 |
| Isoleucine | Leucine | 0.78 |
| N-Acetyl-arginine | N2-Acetyl-lysine | 0.79 |
| N-Acetyl-arginine | N6-Acetyl-lysine | 0.66 |
| N-Acetyl-arginine | Tyrosine | 0.62 |
| N6-Acetyl-lysine | Tyrosine | 0.66 |
| NAD+ | UDP-glucose | 0.69 |
| Phenylalanine | Tyrosine | 0.73 |
| UDP-acetyl-glucosamine | UTP | 0.78 |
| Keto(iso)leucine | Ketovaline | 0.63 |

TABLE 11
The pairs of age-related compounds in RBC that
show relatively high correlation values.

| Age-related | Age-related | Correlation |
|---|---|---|
| 1,5-Anhydroglucitol | Isoleucine | 0.61 |
| 1,5-Anhydroglucitol | Keto(iso)leucine | 0.71 |
| 1,5-Anhydroglucitol | Ketovaline | 0.67 |
| Acetylcarnosine | Keto(iso)leucine | 0.80 |
| Aspartate | Dimethyl-guanosine | 0.62 |
| CDP-choline | CDP-ethanolamine | 0.80 |
| Citrulline | Diphospho-glycerate | 0.76 |
| Citrulline | Glucose-6-phosphate | 0.64 |
| Citrulline | Glutamate | 0.64 |
| Citrulline | Glycerol-phosphate | 0.74 |
| Citrulline | N-Acetyl-arginine | 0.62 |
| Citrulline | N6-Acetyl-lysine | 0.75 |
| Citrulline | S-Adenosyl-homocysteine | −0.64 |
| Citrulline | Tyrosine | 0.69 |
| Creatine | N-Acetyl-arginine | 0.61 |
| CTP | UTP | 0.64 |
| Dimethyl-arginine | Dimethyl-guanosine | 0.68 |
| Dimethyl-arginine | N-Acetyl-arginine | 0.63 |
| Diphospho-glycerate | Fructose-6-phosphate | 0.73 |
| Diphospho-glycerate | Glucose-6-phosphate | 0.85 |
| Diphospho-glycerate | Glutamate | 0.73 |
| Diphospho-glycerate | Glycerol-phosphate | 0.82 |
| Fructose-6-phosphate | Glucose-6-phosphate | 0.94 |
| Glucose-6-phosphate | Glycerol-phosphate | 0.69 |
| Glutamate | Glycerol-phosphate | 0.71 |
| Glycerol-phosphate | N-Acetyl-arginine | 0.65 |
| Glycerol-phosphate | S-Adenosyl-homocysteine | −0.61 |
| Isoleucine | Leucine | 0.84 |
| Isoleucine | Serine | 0.63 |
| Leucine | Serine | 0.65 |
| N-Acetyl-arginine | N2-Acetyl-lysine | 0.77 |
| N6-Acetyl-lysine | Tyrosine | 0.72 |
| Pentose-phosphate | UDP-glucose | 0.78 |
| Phenylalanine | Tyrosine | 0.62 |
| Keto(iso)leucine | Ketovaline | 0.72 |

Experiment of Fasting

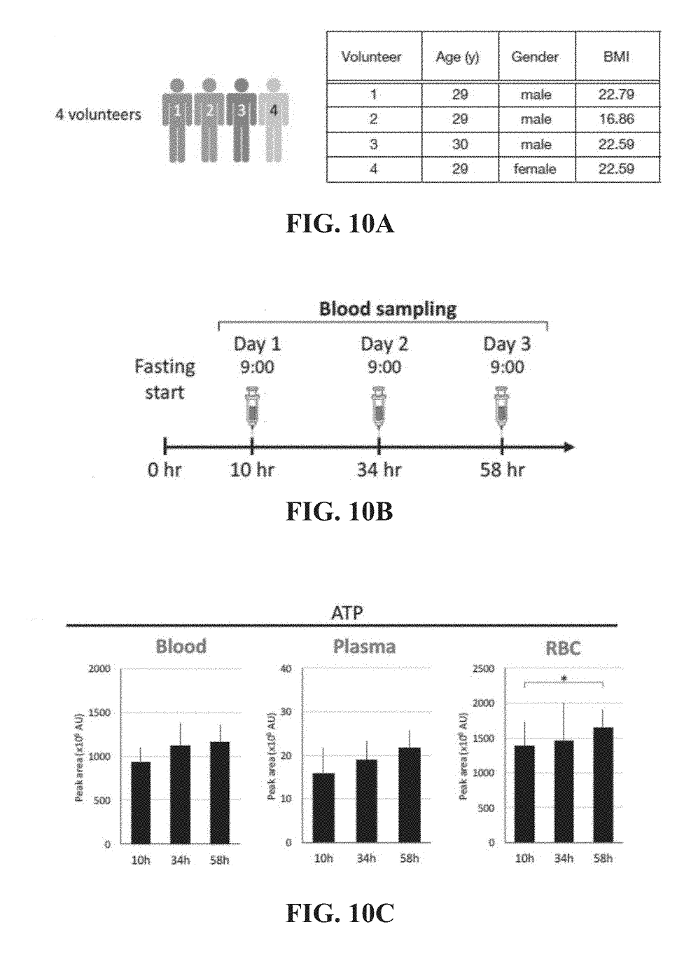

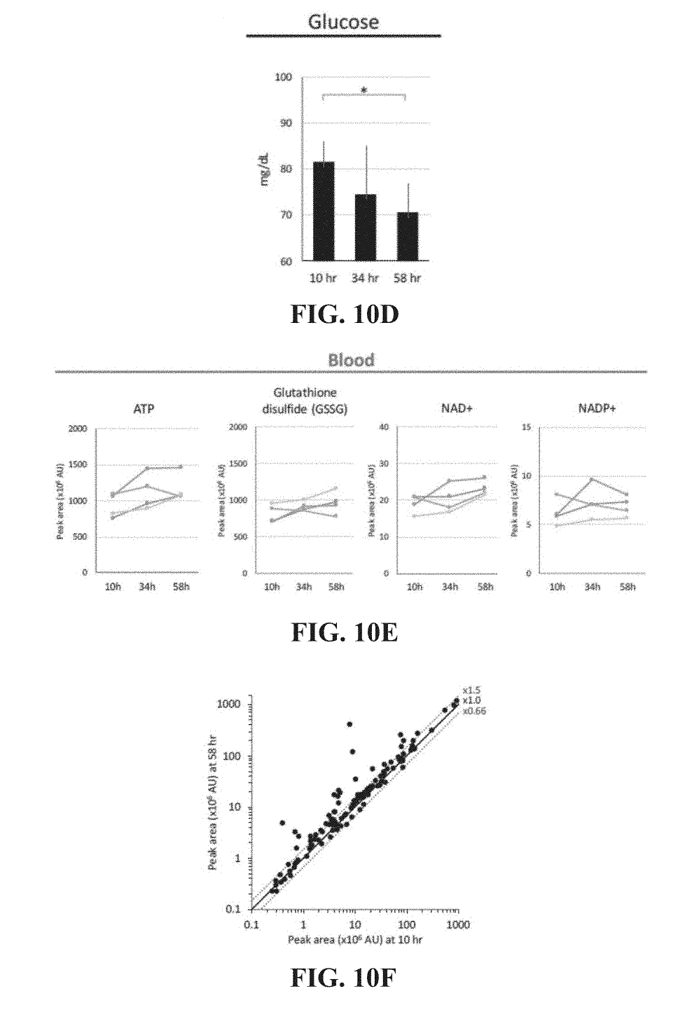

Four healthy, young volunteers (FIG. 10A) fasted for 58 hr. They did not eat or consume any calories, while carrying out their normal routines; however, volunteers imbibed calorie-free drinks. Their blood was sampled on three consecutive weekdays each morning at 9:00 in the laboratory at Kyoto University Hospital (FIG. 10B). Human blood sample preparation and analysis were performed as described above.

Quantification of Blood Metabolites from 4 Volunteers During Fasting

Blood samples were obtained from four young, healthy, non-obese volunteers. Their ages, genders, and BMIs are shown in FIG. 10A. Phlebotomy was performed in the hospital at 10, 34, and 58 hr after fasting, to facilitate rapid preparation of metabolome samples. Immediately after blood collection, metabolome samples for whole blood, plasma, and RBCs were prepared separately, followed by metabolomic measurements by LC-MS. Levels of ATP, an essential energy metabolite, did not change significantly in whole blood, plasma, or RBCs of the four volunteers throughout the fast (FIG. 10C: statistical significance may actually increase slightly in RBCs from 10-58 hr). Plasma ATP levels were much lower than in RBCs or whole blood. All participants remained healthy and manifested no adverse symptoms during the study. Blood glucose levels of participants remained within the normal range (70-80 mg/dl) (FIG. 10D).

Comprehensive, quantitative analyses of blood metabolites were performed. We identified 126 metabolites in human whole blood, approximately half of which were enriched in RBCs described above. During 58 hr of fasting, the majority (62%) of these compounds were maintained at roughly constant levels. For example, levels of essential compounds, such as glutathione, NAD+, and NADP+ remained roughly constant, as in the case of ATP (FIG. 10E).

Fasting-Induced Increases of BCAAs and Ophthalmic Acid

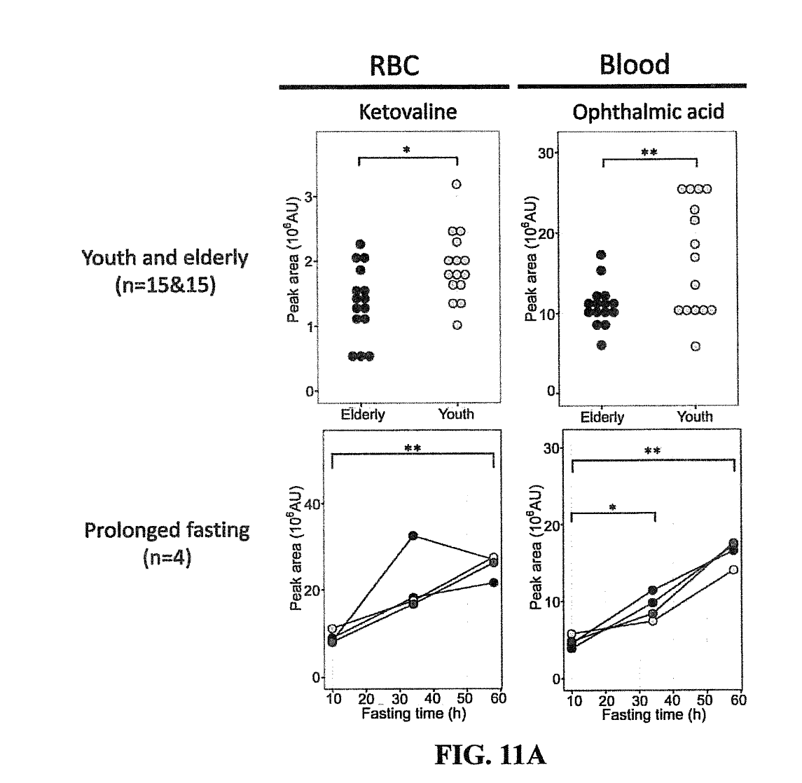

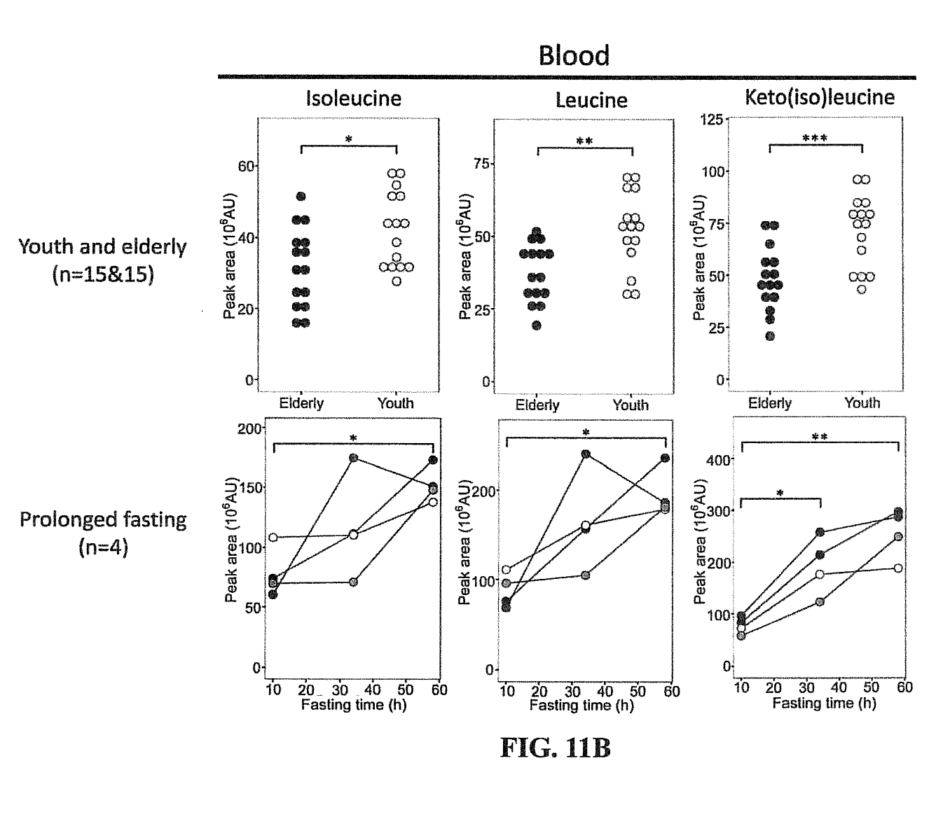

Branched-chain amino acids (BCAAs) are known as fasting markers. Because BCAAs are converted to CoA compounds and used for energy generation via the Krebs cycle, they are implicated in mitochondrial activation. In our analysis, we found a novel BCAA fasting marker, ketovaline, in addition to isoleucine, keto(iso)leucine, leucine, and valine, which were previously known (FIGS. 11A and 11B). These compounds are detected in blood before fasting and the degree of increase after 58 hr of fasting was moderate (2.0-3.4-fold increase). After 58 hr fasting, ketovaline and keto(iso)leucine increased the most (average 3.4-fold) in all four volunteers. Another interesting example is that of a tripeptide analog of glutathione, L-γ-glutamyl-L-α-aminobutyrylglycine, also known as ophthalmic acid (OA). Synthesis of OA employs the same enzymes utilized for glutathione production. Interestingly, the level of OA significantly increased, while that of glutathione remained constant (FIG. 10E).

Interestingly, we found that Keto(iso)leucine, Leucine, Ophthalmic acid, Isoleucine, and Ketovaline. which are lower in elder than in young are upregulated by fasting significantly. Thus, fasting is effective way to treat the subjects to suppress the extent of aging.

In this work, we present a novel method for determining the extent of aging in which a blood metabolite is used as an indicator. The method according to one or more embodiments of the present invention is easy and accurate. We also present a novel method for treating the subject to suppress the extent of aging.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

### REFERENCES

1. Ouchi M, et al. (2012) Effects of sex and age on serum 1,5-anhydroglucitol in nondiabetic subjects. Exp Clin Endocrinol Diabetes 120(5):288-295.
2. Lawton K A, et al. (2008) Analysis of the adult human plasma metabolome. Pharmacogenomics 9(4):383-397.
3. Pitkanen H T, Oja S S, Kemppainen K, Seppa J M, & Mero A A (2003) Serum amino acid concentrations in aging men and women. Amino Acids 24(4):413-421.
4. Mizutani N, et al. (1987) Guanidino compounds in hyperargininemia. Tohoku J Exp Med 153(3):197-205.
5. Leonard J V & Morris A A (2002) Urea cycle disorders. Semin Neonatol 7(1):27-35.
6. Niwa T, Takeda N, & Yoshizumi H (1998) RNA metabolism in uremic patients: accumulation of modified ribonucleosides in uremic serum. Technical note. Kidney Int 53(6):1801-1806.
7. Dungan K M (2008) 1,5-anhydroglucitol (GlycoMark) as a marker of short-term glycemic control and glycemic excursions. Expert Rev Mol Diagn 8(1):9-19.
8. Katsanos C S, Kobayashi H, Sheffield-Moore M, Aarsland A, & Wolfe R R (2006) A high proportion of leucine is required for optimal stimulation of the rate of muscle protein synthesis by essential amino acids in the elderly. Am J Physiol Endocrinol Metab 291(2):E381-387.

What is claimed is:

1. A method for suppressing aging, comprising:
obtaining a blood sample from a subject;
detecting one or more blood metabolites in the blood sample;
identifying the subject as having a biological age greater than a chronological age of the subject by determining metabolite levels of the one or more blood metabolites of the subject; and
subjecting the identified subject to fasting more than 10 hours.

2. The method according to claim 1, further comprising administering a calorie-free food, a calorie-free drink, a hypocaloric food, or a hypocaloric drink to the subject during the fasting.

3. The method according to claim 2, wherein a total caloric intake of the subject is less than 100 kcal during the fasting.

4. The method according to claim 1, wherein identifying the subject is performed by comparing the metabolite levels of the subject with metabolite levels of a control population.

5. The method according to claim 1, wherein the blood sample is at least one selected from the group consisting of whole blood, red blood cells, and plasma.

6. The method according to claim 5, further comprising treating the blood sample with organic solvent at −20° C. to −80° C. immediately after obtaining the blood sample.

7. The method according to claim 6, wherein the one or more blood metabolites comprise at least one metabolite selected from the group consisting of glutathione disulfide (GSSG), UTP, keto(iso)leucine, N-acetyl-arginine, 1,5-anhydroglucitol, acetyl-carnosine, citrulline, dimethyl-guanosine, carnosine, UDP-acetyl-glucosamine, leucine, N2-acetyl-lysine, ophthalmic acid, pantothenate, N6-acetyl-lysine, NAD+, CDP-choline, glycerophosphocholine, histidine, phenylalanine, phosphocreatine, tyrosine, isoleucine, NADP+, pentose-phosphate, S-adenosyl-homocysteine, CDP-ethanolamine, creatine, CTP, fructose-6-phosphate, glycerol-phosphate, serine, tryptophan, UDP-glucose, adenosine, aspartate, dimethyl-arginine, diphospho-glycerate, glucose-6-phosphate, glutamate, glutarate, N-acetyl-(iso)leucine, and ketovaline.

8. The method according to claim 6, wherein the one or more blood metabolites comprise at least one metabolite selected from the group consisting of glutathione disulfide (GSSG), UTP, keto(iso)leucine, N-acetyl-arginine, 1,5-anhydroglucitol, acetyl-carnosine, citrulline, dimethyl-guanosine, carnosine, UDP-acetyl-glucosamine, leucine, N2-acetyl-lysine, ophthalmic acid, pantothenate, N6-acetyl-lysine, NAD+, CDP-choline, glycerophosphocholine, histidine, phenylalanine, phosphocreatine, tyrosine, isoleucine, NADP+, pentose-phosphate, ketovaline, and S-adenosyl-homocysteine.

9. The method according to claim 6, wherein the one or more blood metabolites comprise at least one metabolite selected from the group consisting of glutathione disulfide (GSSG), UTP, keto(iso)leucine, N-acetyl-arginine, 1,5-anhydroglucitol, acetyl-carnosine, citrulline, dimethyl-guanosine, carnosine, UDP-acetyl-glucosamine, leucine, ophthalmic acid, isoleucine, and ketovaline.

10. The method according to claim 6, wherein the one or more blood metabolites comprise at least one metabolite selected from the group consisting of keto(iso)leucine, leucine, ophthalmic acid, isoleucine, and ketovaline.

11. The method according to claim 7, wherein the fasting is performed more than 20 hours.

12. The method according to claim 4, wherein comparing the metabolite levels is performed using a device comprising means for inputting the metabolite levels of the subject and means for determining the extent of aging of the subject.
